(19) United States
(12) Patent Application Publication
(10) Pub. No.: US 2012/0124703 A1
(43) Pub. Date: May 17, 2012

(54) NOVEL COPRINUS COMATUS AND TREMELLA MESENTERICA MUSHROOM STRAINS, PRODUCTS AND EXTRACTS THEREOF AND COMPOSITIONS COMPRISING THEM

(75) Inventor: Solomon P. Wasser, Nesher (IL)

(73) Assignee: PALMED TEVA LTD, Lahavim (IL)

(21) Appl. No.: 13/256,043

(22) PCT Filed: Mar. 10, 2010
(86) PCT No.: PCT/IL2010/000202

§ 371 Date: Jan. 23, 2012

Related U.S. Application Data
(60) Provisional application No. 61/158,837, filed on Mar. 10, 2009.

(51) Int. Cl. A61K 31/716; A61K 31/736; A01N 43/16; A01P 7/04; A01P 1/00; A61P 3/10; A61P 37/02; A61P 3/06; A01H 15/00; C08B 37/00; A61K 31/715; A61K 38/02; A61K 8/96; A61K 8/73; A23L 1/28; A23K 1/16; A61K 36/07

(52) U.S. Cl. 800/297; 424/195.15; 514/54; 536/123.12; 536/123; 424/278.1; 514/1.1; 426/656; 426/655

(57) ABSTRACT

New and distinct varieties of higher Basidiomycetes mushrooms selected from Coprinus comatus HAI-1237 and Tremella mesenterica HAI-17 deposited under The Budapest Treaty with the Centralbureau voor Schimmelcultures (CBS) under Accession Nos. CBS 123401 and. CBS 123296, respectively, biomass and extracts thereof and isolated constituents such as b-glucans, fucogalactans and glucuronoxylomannans are disclosed, and their use as natural food supplements, nutraceuticals, prebiotics, beverage products, cosmetics, pet food, and agricultural insecticidal and anti-plant virus compositions

### TECHNICAL FIELD

The present invention relates to medicinal mushrooms, more particularly to species of genera Coprinus and Tremella mushrooms and to new and distinct strains of higher Basidiomycetes designated Coprinus comatus CBS 123401 and Tremella mesenterica CBS 123296. The invention further relates to fruiting bodies, submerged cultivated mycelial or single cell biomass and extracts from these new strains comprising various biologically active compounds, and their use as human and animal dietary supplements, prebiotics, cosmeceuticals, and in the therapy of several diseases and conditions, as well as anti-phytoviral agents.

### BACKGROUND ART

Mushroom biotechnological products have multibeneficial effects to human welfare, e.g., as food, health tonics and medicine, feed and fertilizers, and to protect and regenerate the environment. Pharmaceutical substances with potent and unique health-enhancing properties were isolated recently from medicinal mushrooms and distributed worldwide. Many of them are pharmaceutical products, while others represent a novel class of dietary supplements or “mushroom nutraceuticals” or “nutriceuticals”, mycochemicals, phytochemicals, and designer food. Several antitumor polysaccharides, such as hetero-β-glucans and their protein complexes (e.g., xyloglucans, and acidic β-glucan containing uronic acid) as well as dietary fiber, lectins, and triterpenoids, have been isolated from medicinal mushrooms.

Higher Basidiomycetes mushrooms contain a large amount of polysaccharides, especially different types of β-glucans. The anti-tumor effects of β-glucans seem to be related to their molecular weight and solubility. Only the low-molecular weight lentinan, for example, shows high anti-tumor activity. Unsurprisingly, soluble β-glucans appear to be stronger immunostimulators than insoluble ones.

Submerged cultivated one-cell biomass and fruiting bodies, of some species of genus Tremella, especially strains of T. mesenterica contain high levels of glucuronoxylomannan and β-glucans, and both the biomass and the purified polysaccharides have been shown to possess hypoglycemic and hypotrygliceridic activity (U.S. Pat. No. 6,383,799; U.S. Pat. No. 6,362,397).

Higher Basidiomycetes mushrooms contain a large amount of polysaccharides, proteins, well-balanced essential amino acids, melanins, lipids comprising essential fatty acids, triterpenoids, antioxidant agents, vitamins, and other biological active substances. Also, dietary fibers belonging to glucans, chitin, and heteropolysaccharides including pectinous substances, hemi-celluloses or polyuronides, are abundant in the tissue of all mushrooms, which are capable of absorbing bile acids or hazardous materials in the intestine, and thus can act as carcinostatics and decrease various kinds of poisoning.

In addition, fungal substances are known as: (i) modulators of NF-κB activation pathway that plays critical roles in a variety of physiological and pathological processes; (ii) antioxidant suitable as supplements in the human diet for preventing or reducing oxidative damage caused by oxidative stress reactions; (iii) immunomodulators; and to affect inflammatory processes.

### SUMMARY OF INVENTION

The present invention is directed to new and distinct varieties of higher

Basidiomycetes mushroom selected from Coprinus comatus HAI-1237 deposited under The Budapest Treaty with the Centralbureau voor Schimmelcultures (CBS) under Accession No. CBS 123401 (hereinafter Coprinus comatus CBS 123401), and Tremella mesenterica HAI-17 deposited under The Budapest Treaty with the Centralbureau voor Schimmelcultures (CBS) under Accession No. CBS 123296 (hereinafter Tremella mesenterica CBS 123296).

In other aspects, the present invention relates to the biomass of the mushrooms of the invention rich in nutraceutical agents and biologically active substances including carbohydrates, proteins rich in essential amino acids, vitamins, lipids rich in essential fatty acids, antioxidant agents and minerals. The biomass can be obtained from the fruiting body or the mycelium of Coprinus comatus CBS 123401 or the mycelium of Tremella mesenterica CBS 123296. The mycelial culture of Tremella mesenterica CBS 123296 is in the form of one-cell biomass.

In a further aspect, the present invention relates to extracts from the mushrooms of the inventions having nutraceutical and biological activity. The extracts can be obtained from the mycelium or the fruiting body of the mushrooms.

In still other aspects, the invention relates to novel carbohydrates isolated from the extracts, compositions comprising the biomass or extract from the mushrooms of the invention or the novel carbohydrates isolated from the extracts, to natural food supplement, pharmaceutical, prebiotic, nutraceutical, beverage or cosmetic products comprising a composition of the invention, to a pharmaceutical composition comprising a pharmaceutically acceptable carrier and an active ingredient selected from a composition of the invention and one of the novel carbohydrates, and to processes for producing the biomass and extracts.

### BRIEF DESCRIPTION OF DRAWINGS

FIG. 1 depicts a scheme of the general methodology for the production of submerged cultured mycelium of Coprinus comatus or Tremella mesenterica in fermentor or in bioreactor technology: a—preparation of standard agar media for Petri dish; b—spores or parts of fruiting body which are used for preparation of culture; c—culture on Petri dish; d—museum culture on agar slant in tube; e—microscopic examination of museum culture; f—pre-inoculums culture in 250 mL Erlenmeyer flask; g—homogenization of pre-inoculums culture; h—cultivation of homogenized mycelial biomass in 2 L Erlenmeyer flasks; i—homogenization of mycelial biomass for inoculation fermentor medium; j—growth medium for fermentor; k—cultivation of mycelial biomass in fermentor; l— harvest of mycelial biomass; m—dried biomass formulations for dietary supplements (DS), pharmaceuticals and other products; HM, Harvest Mycelia;

FIG. 2 shows effect of E1 and E2 extracts on pIκBα levels as determined by Western immunoblot. The figure is representative of two independent experiments with similar results. E1—Cultural liquid (water) extract; E2—Ethyl acetate extract.

FIG. 3 displays densitometric analysis of the Western immunoblot of FIG. 2 showing the effects of E1 and E2 extracts on pIκBα compared to 100 μM H₂O₂ effect. (Results are presented as folds of two independent experiments to the mean of only H₂O₂-treatments±standard deviation).

FIG. 4 shows IKKβ inhibition activity of Coprinus comatus extracts E1 and E2. Extracts E1 and E2 (100 and 200 μg/ml) and 600 nM of the IKK-β inhibitor Fuct (1 μM) were incubated with 100 ng of GST-IκBα substrate and 5 ng of IKK-β enzyme, and tested for their ability to inhibit IKK-β activity using an ELISA-based kinase activity assay as described in Materials and Methods. E1—crude cultural liquid (water) extract; E2—crude ethyl acetate extract.

FIG. 5 depicts ¹H NMR spectrum of the Coprinus water extract.

FIGS. 6A-D show Gas Chromatography (GC) analysis of monosaccharide content of the whole cells, extracts, and residues after extraction. (A) Water extract; (B) Whole cells; (C) Cells after water extraction; and (D) Cells after NaOH extraction.

FIG. 7 depticts a size-separation chromatogram of polysaccharides from water and NaOH extracts of Coprinus comatus, partially separated by size-exclusion chromatography on Sephadex G-50.

FIGS. 8A-B show methylation analysis of β-glucan extracted from Ganoderma sp. (A) and Coprinus comatus CBS 123401 (B).

FIG. 9 depicts a GC trace of methylated sugars obtained from β-glucan isolated from Tremella mesenterica CBS 123296.

FIG. 10 show GC traces of methylated sugars obtained from the newly isolated strain of Tremella mesenterica CBS 123296 (A) and pure glucuronoxylomannan (B).

FIG. 11 shows the effect of GXM (1000 μg/mL) on perceptivity of the tobacco plant of variety Immune 580 relative to Tobacco Mosaic Virus (TMV). Abscissa: interval between introduction of GXM and inoculation of TMV (days). Ordinate: the ratio (%) of the number of local lesions in the experiment (dark solid bars) and in control (textured light bars).

FIG. 12 shows the influence of GXM (2500 μg/ml) on perceptivity of the Nicotiana tabacum plant of variety Immune 580 relative to TMV. Abscissa: interval between introduction of GXM and inoculation of TMV (days). Ordinate: the ratio (%) of the number of local lesions in the experiment (dark solid bars) and in control (textured light bars).

FIG. 13 shows the influence of GXM (2500 μg/ml) on the growth of local lesions, induced by TMV in the Nicotiana tabacum plant of variety Immune 580. Abscissa: interval between introduction of GXM and inoculation of TMV (days). Ordinate: size of local lesions (mm) in the experiment (dark solid bars) and in control (textured light bars).

FIG. 14 shows the influence of actinomycin D (AMD) on induced GXM resistance in Nicotiana tabacum plants of variety Immune 580 inoculated with TMV. Abscissa: variants of the experiment: 1—GXM; 2—mixture of GXM and AMD (10 μg/mL); 3—AMD (10 μg/mL) introduced 2 days after GXM; 4—AMD (20 μg/mL) introduced 2 days after GXM; 5—AMD (10 μg/mL). Ordinate: the ratio (%) of the quantity of local lesions in the experiment (dark solid bars) and in control (textured light bars).

### DETAILED DESCRIPTION OF THE INVENTION

In one aspect, the present invention relates to a biomass of the Basidiomycetes mushrooms Coprinus comatus CBS 123401 and Tremella mesenterica CBS 123296 rich in nutraceutical agents and biologically active compounds including proteins rich in essential amino acids and carbohydrates and further comprising vitamins, lipids rich in essential fatty acids, antioxidant agents, and minerals.

In one embodiment, the biomass is obtained from the fruiting body or the mycelium of Coprinus comatus CBS 123401 or the mycelium of Tremella mesenterica CBS 123296 in the form of single-cell biomass, for example by cultivation of the strain in submerged culture on nutrient media.

The invention further relates to pure submerged mycelial cultures of Coprinus comatus CBS 123401 and Tremella mesenterica CBS 123296, wherein the mycelial culture of Tremella mesenterica CBS 123296 is in the form of single-cell biomass. The fact that the Tremella mesenterica mycelial culture grows as a unicellular organism, like yeast, provides for a very high growth rate and biomass productivity which is the highest among all Basidiomycetes mushrooms, reaching up to 27 g of dried biomass per liter of media.

The chemical composition of mycelium of the two mushrooms of the invention was determined as shown in Examples 4 and 7. Many of the constituents shown herein to be present in the mushrooms of the present invention have multiple beneficial properties such as anti-cancer activity, immunomodulating activity, anti-glycemic, anti-diabetic and insecticidal activity.

The mycelial biomass of Coprinus comatus CBS 123401 has about 39% carbohydrates and about 37% proteins, and that of Tremella mesenterica CBS 123296 has about 54% carbohydrates and about 20% proteins of the dry weight of mycelium. Submerged culturing of mushroom polysaccharide producers allows the production under controlled conditions of a constant composition in a short time period using culture medium of defined composition.

The carbohydrates in the biomass include both polysaccharides and di- and mono-saccharides. Examples of polysaccharides of the biomass of Coprinus comatus CBS 123401 include β-glucans, preferably a low molecular weight water-soluble β-glucan, and galactans, preferably neutral fucogalactan.

Thus, the present invention relates to a novel low molecular weight water-soluble β-glucan composed of a backbone structure of β-1-3-linked D-glucose residues bearing, at some of the 6-positions, side chains of β-1-6-D-glucose residues as shown in Example 4.2 herein. The β-glucan is obtained from Coprinus comatus, preferably Coprinus comatus CBS 123401, and has a molecular weight of less than 10,000 Da, preferably about 1000 to about 10,000 Da.

Examples of polysaccharides of the biomass of Tremella mesenterica CBS 123296 include β-glucans, preferably a linear 3,4 β-glucan, and glucuronoxylomannan. Thus, the present invention further relates to a novel water insoluble linear 3,4 β-glucan and to a glucuronoxylomannan obtained from Tremella mesenterica CBS 123296 as shown in Example 7.4 herein. The glucuronoxylomannan consists of a linear backbone of α-(1→3)-linked mannan, glycolized by β-(1→2)(1→4)-linked oligosaccharides of xylose and glucuronic acid, which bestowes polyanion properties, and may by used as an anti-glycemic and anti-diabetic agent.

The β-glucans of the present invention have antitumor and immunomodulating, particularly immunostimulatory activities, and the glucuronoxylomannan of the present invention has hypoglycemic, immunostimulating, and hypocholesterolemic activities and are promising as a herbal medicine to prevent and treat diseases and conditions in which strengthening of the immune system is important, such as to prevent and treat diabetes, cancer, viral diseases such as AIDS, heart diseases, blood pressure, and as hypocholesterolemic agents to treat high cholesterol conditions. Polysaccharides of both species can be a source of new prebiotics. A “prebiotic agent” is defined herein as a selectively fermented ingredient that allows specific changes, both in the composition and/or activity, in the gastrointestinal microflora that confers benefits upon host well-being and health. Chitin, also present in the biomass, is an important constituent of dietary fibers.

The mono- and di-saccharides found in the mycelial biomass include glucose arabinose, xylose, mannose, galactose, glucosamine and trehalose. All these mono- and di-saccharides are important for the health. In addition, mannose has been shown to prevent the adhesion of bacteria to tissues of the urinary tract and bladder, and glucosamine is known as useful for treatment of osteoarthritis and to rebuild cartilage.

The mycelial biomass proteins of the mushrooms of the present invention are rich in glutamic acid, aspartic acid, leucine, cystein, methionine, threonine, valine, isoleucine, leucine, tyrosine, phenylalanine, lysine and histidine. C. comatus further contains γ-aminobutyric acid. Thus, the proteins of mycelium contain 10 out of the 11 essential amino acids; threonine, valine, isoleucine, leucine, histidine, lysine, methionine, cysteine, phenylalanine, tryptophan and tyrosine. The biomass of the present invention therefore constitutes an important dietary supplement due to the presence of the proteins rich in essential amino acids.

The biomass of Coprinus comatus CBS 123401 comprises the vitamins: A, B₁, B₂, B₃, C, and E; and the biomass of Tremella mesenterica CBS 123296 comprises the vitamins: A, B₁, B₂, B₃, B₆, B₇, C, and E. Thus, the biomass and extracts of the mushrooms of the present invention containing high levels of important vitamins serve as an excellent source of vitamins and may be used as nutraceuticals and/or may be added to food and beverage products as dietary supplements.

The mycelial biomass of Coprinus comatus CBS 123401 further comprises lipids including the fatty acids pentadecanoic, palmitic, palmitoleic, heptadecanoic, stearic, oleic, linoleic (C18:2n6), α-linolenic (C18:3n3), γ-linolenic (C18:3n6), arachidic, heneicosanoic, behenic, and lignoceric acids; and the biomass of Tremella mesenterica CBS 123296 comprises the fatty acids oleic acid-(C18:1), linoleic acid (C18:2n6), palmitic acid (C16:0), palmitoleic acid (C16:1), stearic acid (C18:0) and myristic acid. The fatty acids are found in the mushroom in the form of their esters with glycerol. A high nutritional quality of the mushroom is made evident by the presence of the essential unsaturated fatty acids α-linolenic acid (C18:3n3) and linoleic acid (C18:2n6). The latter gives rise to the omega-6 series of polyunsaturated fatty acids, which incorporation into phospholipids affect cell membrane properties such as fluidity, flexibility, permeability and the activity of membrane bound enzymes.

The mycelial biomass of the mushrooms of the invention comprises also minerals, both macroelements and microelements, including aluminum, copper, iron, potassium, magnesium, manganese, phosphorus, silicon, sodium, titanium and zinc. A daily dose of biomass of either one of the two mushrooms of the present invention endows an excellent source of iron and other minerals.

It has also been found in accordance with the present invention that the mycelial and biomass of the Coprinus comatus CBS 123401 strain of the invention comprise anti-oxidant agents, free-radical scavenging agents, melanin (confers protection against photo-aging of the skin, particularly protects the skin from solar UV radiation, and also protects against damage to internal organs caused by ionizing radiation, and may serve to sequester potentially toxic metal ions through its carboxylate and phenolic hydroxyl groups) and lectins (see Example 4.3), in particular lactose, galactose and glucosamine binding lectins, which can be useful in assays involving the identification of sugar moieties of polysaccharides and glycoproteins.

The biomass of the mushrooms of the present invention may be further used in prebiotic or nutraceutical compositions. A “mushroom nutraceutical” is defined as a refined or partially refined extract or dried biomass from either the mycelium or the fruiting body of the mushroom, which is consumed in the form of capsules or tablets as a dietary supplement (not a conventional food) and which has potential therapeutic applications. Regular intake may enhance the immune responses of the human body, thereby increasing resistance to disease, and in some cases causing regression of a disease state.

In another aspect, the present invention provides extracts of the mushrooms of the invention having nutraceutical and biological activities. In one embodiment, the extract is from Coprinus comatus CBS 123401. In another embodiment extract is from Tremella mesenterica CBS 123296. The extracts are obtained from the fruiting body or the mycelium of Coprinus comatus CBS 123401 or from the mycelium of Tremella mesenterica CBS 123296. In a preferred embodiment, the extracts of the mushrooms of the invention are obtained from a pure submerged mycelium culture.

The extract obtained from Coprinus comatus CBS 123401 culture is enriched in a low molecular weight water-soluble β-glucan and/or galactans, preferably neutral fucogalactan, and the extract obtained from Tremella mesenterica CBS 123296 culture, is enriched in a linear 3,4 β-glucan and/or glucuronoxylomannan, which has anti-glycemic and anti-diabetic activity.

In one embodiment, the biological activity present in the extract of the mushrooms of the present invention is NF-κB pathway modulating activity, anti-oxidant activity, free radical scavenging activity, anti-radiation activity, metal ion scavenging activity, interferonogenous activity, immunomodulating activity, anti-glycemic activity, anti-diabetic activity, hypocholesterolemic activity, anti-allergic activity, anti-parasitic activity, insecticidal activity and/or anti-plant viral activity.

The terms “interferonogenous activity” and “interferonogenous agent” as used herein refer to an activity or agent that increase the concentration of interferon in the blood plasma of a mammal.

The terms “immunomodulating activity” and “immunomodulating agent” as used herein refer to, but are not limited to, mitogenicity, stimulation of hematopoietic stem cells, activation of alternative complement pathway, and activation of immune cells such as T_{H} cells, Tc cells, B cells, macrophages, dendritic cells, and natural killer (NK) cells.

The terms “anti-glycemic activity” and “anti-glycemic agent” refer to an activity or agent that reduces blood glucose level, while the terms “anti-diabetic activity” and “anti-diabetic agent” refer to an activity or agent that treats diabetes mellitus by lowering glucose levels in the blood.

The insecticidal activity involves attracting social insects such as carpenter ants, fire ants, coptotermes, Formosan termites and reticulitermes termites and infecting and killing these insects.

In particular, and as is shown herein below in the Examples, Coprinus comatus CBS 123401 ethyl acetate extract has NF-κB pathway modulating activity (Example 3), and anti-oxidant activity and/or free radical scavenging (Example 2). Such an extract may be useful in treatment of an NF-κB-dependent disease such as, but not limited to, cancer, immunological disorders, septic shock, transplant rejection, radiation damage, reperfusion injuries after ischemia, arteriosclerosis and neurodegenerative diseases.

The extract obtained from Tremella mesenterica CBS 123296 culture comprises glucuronoxylomannan and therefore has anti-diabetic activity, and further comprises immunomodulating activity, for example such activity that causes an increase of functional reserve of macrophages, and interferrouneus activity (see Example 8) and/or anti-plant viral activity (see Example 9). The term “functional activity” as used herein refers to the difference between the spontaneous and stimulated (NBT-tests) activity indices, i.e. the activity of a phagocytotic cell, such as a macrophage, at its resting state as compared with its activity following activation by for example exposure to a pathogenic bacterium.

In still another aspect, the present invention relates to a composition comprising a biomass or extract according to the present invention. In certain embodiments, the composition comprises a mixture of biomasses obtained from Coprinus comatus CBS 123401 and Tremella mesenterica CBS 123296, or a mixture of extracts obtained from Coprinus comatus CBS 123401 and Tremella mesenterica CBS 123296. In one embodiment, the composition comprises a biomass rich in nutraceutical agents and biologically active substances obtained from the mycelium or from the fruiting body of Coprinus comatus CBS 123401, or an extract of said biomass. In another embodiment the composition comprises a biomass rich in nutraceutical agents and biologically active substances obtained from the mycelium of Tremella mesenterica CBS 123296, or an extract of said biomass.

In still yet another aspect, the present invention relates to a composition comprising a carbohydrate selected from the low molecular weight water-soluble β-glucan, the water insoluble linear 3,4 β-glucan, the glucuronoxylomannan, all of which as defined herein above and in the Examples herein below, or a combination of at least two of these carbohydrates. In view of the above, the present invention provides, in an additional aspect, natural food supplement, prebiotic, nutraceutical, beverage and cosmetic products comprising a composition of the present invention. The present invention further provides pet food, insecticidal, anti-parasitic and anti-plant virus products, comprising a composition of the present invention.

In yet an additional aspect, the present invention provides a pharmaceutical composition comprising a pharmaceutically acceptable carrier and an active ingredient selected from (a) a composition of the present invention; (b) the low molecular weight water-soluble β-glucan; (c) the water insoluble linear 3,4 β-glucan; (d) the glucuronoxylomannan; all of which as defined herein above and in the Examples herein below; or (e) a combination of at least two of the active ingredients of (b) to (d).

In certain embodiments, the natural food supplement, prebiotic or a nutraceutical product and the pharmaceutical compositions of the present invention may be used for (a) treating diabetes or reducing blood glucose levels; (b) inducing an immunomodulatory response; or (c) reducing blood cholesterol levels or reducing the build up of cholesterol.

The natural food supplement, prebiotic or nutraceutical product, or a pharmaceutical composition of the present invention may be administered alone or in combination with an anti-cancer drug, to a cancer patient in order to induce an immunostimulatory response for treating cancer.

The term “treating” as used herein refers to the alleviation, reduction of progression or complete cure of the disease or disorder, or to the reduction of symptoms related to or caused by the disease or disorder.

The pharmaceutical compositions for use in accordance with the present invention may be formulated in conventional manner using one or more pharmaceutically acceptable carriers comprising excipients and auxiliaries. Techniques for formulation and administration of drugs may be found, for example, in “Remington's Pharmaceutical Sciences”, Mack Publishing Co., Easton, Pa., latest edition.

The pharmaceutical compositions of the present invention are formulated for systemic administration by any suitable route, for example, for oral delivery, parenteral delivery including intramuscular, intravenous, subcutaneous, intrathecal, or intraperitoneal injection, or for local administration by topical drug delivery.

For any composition for use in the method of the invention, the therapeutically effective amount or dose can be estimated initially from in-vitro and cell culture assays. For example, a dose can be formulated in animal models to achieve a desired concentration or titer. Such information can be used to more accurately determine useful doses in humans.

In still an additional aspect, the present invention relates to an agricultural composition comprising an agricultural carrier and an active ingredient selected from a composition of the present invention or the glucuronoxylomannan as defined herein. In certain embodiments, the agricultural composition is for inducing resistance in plants to a plant pathogen, such as a plant virus.

In one embodiment, the plant-virus is a plant virus capable of inducing a hypersensitive response in the infected plant, e.g. plant viruses including Tobacco mosaic virus and other Tobamoviruses, such as tomato mosaic virus, pepper green mottle virus and ondontoglossum ringspot virus. In certain embodiments, the virus is Tobacco mosaic virus.

The agricultural compositions of the invention may further comprise inert additives. Such additives include thickeners, flow enhancers, wetting agents, antifoaming agents, buffers, lubricants, fillers, drift control agents, deposition enhancers, adjuvants, evaporation retardants, frost protecting agents, insect attracting odor agents, UV protecting agents, fragrances, and the like. The thickener may be a compound that is soluble or able to swell in water, such as, for example, polysaccharides of xanthans (e.g., anionic heteropolysaccharides), alignates, guars or celluloses; synthetic macromolecules, such as polyethylene glycols, polyvinyl pyrrolidones, polyvinyl alcohols, polycarboxylates of swellable structure-forming silicates such as pyrogenic or precipitated silicic acids, bentonites, montmorillonites, hectonites, or attapulgites; or organic derivatives of aluminum silicates. The frost protecting agent may be, for example, ethylene glycol, propylene glycol, glycerol, diethylene glycol, triethylene glycol, tetraethylene glycol, urea, or mixtures thereof. The antifoaming agent may be, for example, a polydimethylsiloxane. The agricultural composition may also comprise surfactant systems adapted to water- or oil-based products, as is commonly known in the art.

The present invention further provides a process for producing an extract from the mushrooms of the present invention having biological activity, wherein the biological activity is NF-κB pathway modulating activity, anti-oxidant activity, free radical scavenging activity, anti-radiation activity, metal ion scavenging activity, interferonogenous, immunomodulating, anti-glycemic, anti-diabetic, hypocholesterolemic activity, anti-allergic activity, anti-parasitic activity, or anti-plant viral activity, said process comprising: cultivating the fungi Coprinus comatus CBS 123401 or Tremella mesenterica CBS 123296 in submerged culture in nutrient media, isolating the resulting biomass of edible fungi from the culture broth, drying and grinding said biomass into fine powder which is subjected to solvent extraction and freeze drying. In particular, the process is for producing an extract of Tremella mesenterica, preferably Tremella mesenterica CBS 123296, enriched in glucuronoxylomannan.

A further process is provided for producing a biomass from the mushrooms of the present invention which is rich in polysaccharides, monosaccharides, proteins, essential amino acids, vitamins, essential fatty acids, minerals and microelements, said process comprising: cultivating said mushrooms in submerged culture on nutrient media, isolating the resulting biomass of edible fungi from the culture broth, and drying and grinding said biomass into fine powder.

Of particular importance is the further process provided by the present invention for cultivating on nutrient media a single cell submerged culture of a mushroom comprising the genus Tremella selected from Tremella mesenterica, Tremella fuciformis, and Tremella aurantia, preferable Tremella mesenterica CBS 123296.

It has been found in accordance with the present invention that the nutrient media used for cultivating Coprinus comatus for the purpose of the processes defined above should be of the following composition (g/L of distilled water): glucose, 15; peptone, 3; yeast extract, 5; KH₂PO₄, 0.8; K₂HPO₄, 0.2; MgSO₄.7H₂O, 0.5; and the nutrient media used for cultivating Tremella mesenterica for the purpose of the processes defined above is of the following composition (g/L of distilled water): Sucrose, 50; yeast extract, 0.5; KCl, 1; Mg acetate.4H₂O, 1.0; NaH₂PO₄.H₂O, 0.5; Na₂HPO₄.7H₂O, 1.0.

The invention will now be illustrated by the following non-limiting Examples.

### EXAMPLES

### Materials and Methods

1. Submerged Cultivation of Mycelial Biomass of Coprinus comatus in Erlenmeyer Flasks and Fermentor

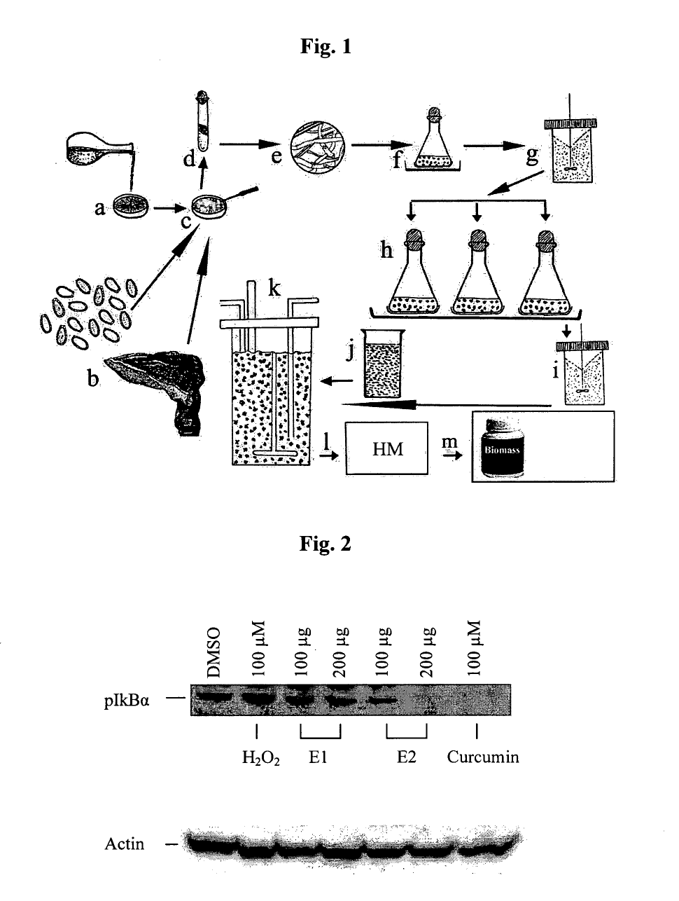

FIG. 1 shows the general methodology for the production of submerged cultured mycelium of Coprinus comatus CBS 123401 and Tremella mesenterica CBS 123296 using fermentor or bioreactor technology.

The general scheme of mushroom submerged culture mycelium (SCM) production includes 5 steps of culture growth:

Museum culture (I)->Intermediate culture (II)->Pre-inoculums culture (III)->Inoculums culture (IV)->Fermentation culture (V).

Three types of culture media are used for SCM production: standard agar medium (steps I and II), liquid standard inoculums medium (steps III and IV), and fermentation medium (step V). Museum cultures are, developed on agar slants in tubes; intermediate cultures are developed on agar slants in tubes or Petri dishes. Pre-inoculums and inoculums cultures are developed in Erlenmeyer flasks using a rotary shaker. Fermentation cultures are developed in fermentor Bioflo 2000 (New Brunswick Scientific, USA) that is equipped with instrumentation for the measurement and/or control of agitation, temperature, pH, dissolved oxygen concentration (pO₂), and foam.

For the first pre-inoculums culture, 250 ml Erlenmeyer flask is inoculated by one to three week old mushroom mycelium from the Petri dish. Five-to-six pieces (5-7 mm in diameter) from mycelium growing on the edge of the agar plate were transferred into the Erlenmeyer flask and cut on the flask wall into small pieces to increase the number of growth points of mycelia. Mycelium was inoculated in 250-mL Erlenmeyer flasks filled with 100 ml of defined synthetic medium. Fungal inocula were grown on synthetic medium consisting of the following components (g/L of distilled water): (g l⁻¹): glucose, 15; peptone, 3.0; yeast extract, 5.0; KH₂PO₄, 0.8; K₂HPO₄, 0.2; MgSO₄.7H₂O, 0.5. Initial pH of the media was 6.0. Phosphate salts were sterilized separately (Sigma-Aldrich, St Louis, Mo., USA). The cultivation of inoculated flasks is carried out on a rotary shaker at 100 rpm and 27° C. for 6-7 days. At the end of cultivation, 1 ml of sample is taken from the culture for microscopic observation of culture purity.

For the second inoculums culture the biomass from the first pre-inoculums culture (pellets) was homogenized 2×30 seconds using a Waring Laboratory Blender (Waring, USA) and inoculated in a 2 L flask containing 700 mL of the same medium.

After 5-7 days of cultivation, mycelial biomass (pellets) were homogenized and used as inoculums culture for growth in a fermentor (Bioflo 2000 10 L, New Brunswick Scientific, USA) with 10 L of working volume on the same synthetic medium mentioned above. Initial parameters of cultivation were as follows: temperature 27° C.; pH—6.1; agitation—100 rpm, aeration—0.2 v/v/min. Antifoam used was polypropylene glycol 2000; 4% NaOH and 4% HCl were used to control pH.

TABLE 1
Mycelial biomass production of Coprinus comatus CBS 123401 in
submerged culture as a function of time

| | | | Aeration, | | |
|---|---|---|---|---|---|
| Time, h | pH | DO, % | v/v/min | RPM | Biomass, g/l |
| 0 | 6.14 | 100 | 0.2 | 100 | 0.2 |
| 24 | 5.8 | 28 | 0.2 | 100→200 | 0.8 |
| 48 | 5.8→6.0 | 19 | 0.2→0.4 | 300 | 3.5 |
| 72 | 6.0 | 17 | 0.4→0.5 | 300 | 5.0 |
| 96 | 6.0 | 14 | 0.5 | 300 | 6.9 |
| 120 | 6.0 | 18 | 0.5 | 300 | 7.5 |
| 144 | 6.0 | 20 | 0.5 | 300 | 8.4 |
| 168 | 6.0 | 22 | 0.5 | 300 | 9.3 |

DO, dissolved oxygen

Initially, pH of the medium was not controlled. However, when it decreased to 5.2, the pH was kept constant automatically at the level of 6.0 to favor the fungus growth. After 24 h, the speed of agitation was increased to 200 rpm, then after 48 h to 300 rpm. After 48 h, the rate of aeration of the medium was increased to 0.4, then (after 72 h) to 0.5 v/v/min.

The maximal yield of mycelial biomass was 93 g/L of wet biomass or 9.3 g of dry biomass achieved on day 7 of fungus cultivation. The conditions of the cultivation are defined in Table 1. Vacuolated hyphae with clamp connections of mycelial biomass of Coprinus comatus CBS 123401 can be seen after 7 days of fungus cultivation (not shown).

2. Evaluation of Coprinus comatus Antioxidant Activity

Biomass Estimation. After 8-11 days of C. comatus submerged cultivation, mycelial biomass was harvested with filtration and dried at 50° C. to a constant weight. The dried mycelia were milled to a powder form for extraction.

Antioxidant Extraction from C. comatus Biomass. Harvested mushroom mycelia were dried at 50° C. and milled to powders (4-10 g). Three different solvents (culture liquid instead of water, ethanol, and ethyl acetate) were used to extract antioxidant compounds from mushroom mycelia in ascending polarity. Although there were no literature data on the antioxidant presence in the culture liquid during Basidiomycetes cultivation, it is supposed that these mushrooms are capable to accumulate these compounds extracellularly. Therefore, to correctly evaluate the total antioxidant activity of screened mushrooms, it was decided to use proper culture liquids instead of water for the antioxidant extraction from the fungal biomasses.

In the first stage the mycelium was extracted for 3 h with culture liquid (1 g/10 ml) at 80° C. (using a water bath). After extraction, insoluble compounds were separated by centrifugation at 6000 rpm for 15 min and filtrated through the Wathman filter paper N 4. Filtrates were evaporated. The residues after centrifugation were then successively extracted on the rotary shaker at 150 rpm with ethanol (80%) at 27° C. and 3 h. After extraction the solutions were centrifuged, filtrated, and the organic solvents were evaporated from the extracts.

Antioxidants Extraction from Culture Liquid of Coprinus comatus CBS 123401. After biomass filtration, pH of the culture growth media was decreased to 2.0 using 96% sulphuric acid. One liter of growth medium per strain was separated 3 times with 500 ml of ethyl acetate, and the extract-solvent mixture was washed once with 0.5 l distilled water using a glass chemical separator. The extract-solvent mixtures were left in a chemical hood for solvent evaporation till a resin (or powder) was formed, which represented the actual crude fungal extract, collected in previously weighed 4 ml plastic tubes. All extracts were diluted with 99.9% dimethyl sulphoxide (DMSO) to the final concentration of 50 mg/ml, distributed into Eppendorf tubes, and kept at 70° C. prior to use.

### Antioxidant Activity Assays

β-Carotene Bleaching Method. The antioxidant activity of C. comatus extracts was determined according to the β-carotene bleaching method. A reagent mixture containing 1 ml of (3-carotene (Sigma) solution (0.2 mg/ml in chloroform), 0.02 ml of linoleic acid (Sigma), and 0.2 ml of Tween 80 (Sigma) was evaporated to dryness under a nitrogen stream. Fifty milliliters of oxygenated distilled water and 0.2 ml of mushroom crude extracts (either ethanol or culture liquid) with different concentrations (2-8 mg/ml) were added. Pure methanol or water (0.2 ml) was used as the control, and the blank contained all the earlier chemicals except β-carotene. All these mixtures were then shaken to form a liposome solution and then incubated at 50° C. for 2 h. The absorbance of an aliquot (1 ml) of these liposome solutions at 470 nm was monitored by a spectrophotometer at time intervals of 20 min. Butylated hydroxyanisole (BHA) (Sigma) (2 mg/ml in methanol) was used as the standard. The bleaching rate (R) of f3-carotene was used as the standard. The bleaching rate (R) of 13-carotene was calculated according to

Equation (1)

R=ln(a/b)/t Equation (1)

where: ln—natural log, a—absorbance at time 0, b—absorbance at time t, and t—incubation interval 20, 40, 60, 80, 100, or 120 min.

The antioxidant activity (AOA) was calculated, in terms of percent inhibition relative to the control, using Equation (2)

AOA=[(Rc_{control}−Rₛₐₘₚₗₑ)/R_{control}]×100 Equation (2)

Scavenging Activity on 1,1-diphenyl-2-picrylhydrazyl. The scavenging activity of extracts from C. comatus was measured on 1,1-diphenyl-2-picrylhydrazyl (DPPH) radicals. An aliquot of 0.5 ml of 0.1 mM DPPH radical (Sigma) in methanol was added to a test tube with 1 mL of mushroom ethanol or water extract of different concentrations (0.5 to 9 mg/mL). Methanol or water was used instead of the mushroom sample as a control. The reaction mixture was vortex-mixed at room temperature and the absorbance was determined immediately after mixing by measuring at 520 nm with a spectrophotometer. Inhibition of free radicals by DPPH was calculated as follows: I (%)=(A_{blank}−Aₛₐₘₚₗₑ/A_{blank})×100, where I is inhibition (%), A_{blank} is the absorbance of the control reaction (containing all reagents except the test compound), and Aₛₐₘₚₗₑ is the absorbance of the test compound. The extract concentration providing 50% inhibition (EC₅₀) was calculated from the graph of scavenging activity of radical percentage against extract concentration. Butylated hydroxyanisole (BHA) in concentration of 1 mg/ml methanol was used as the standard. Each value is expressed as mean±standard deviation (n=3).

3. Study on Biological Activity of Extracts from Submerged Coprinus comatus

Cell Culture Preparation for the Experimental Work. All experimental work with cancer cell cultures was carried out in a sterile biological hood. Prior to each experiment, cells were trypsinized, collected, and counted in order to seed proper cell amounts according to the needs of the provided experiments.

MCF7 breast cancer cell culture normally grows attached to the flask bottom. In order to prepare these cells for use, the medium was discarded from the flask using disposable pipettes and 1-2 ml of trypsin was then added for 2-3 min. During trypsinization, cells were kept in a humidified incubator at 37° C. After cells detached from the flask bottom, in order to stop the trypsinization process, 5-10 ml of fresh medium was added, depending on the size of the flask used. Cells were mixed well via multiple pipetting, and 100 μl of the cell suspension were stained with 100 μl of 0.4% trypan blue solution, mixed well, and counted under microscope using a hemacytometer following the trypan blue exclusion method.

After establishing the available cell number, the cell suspension was additionally diluted and a certain number of cells were seeded according to the requirement of each experiment. Some cells were always kept in order to re-grow and sustain the available cell line for further experimental work. Stock suspensions of all cell lines were kept in liquid nitrogen and the experimental cell cultures were refreshed once every 2-3 months.

Cell Lysis, Preparation of Total Cell Lysates. In order to measure the intracellular levels of certain proteins as a response to the crude fungal extracttreatments, each time after cells were collected, a total cell lysis was performed. The exact procedure of the total cell lysis is described below.

Cells were collected and washed 3 times with cold phosphate buffered saline (PBS) through centrifugation for 5 min, at 3,000 rpm, and 4° C. The final cell amounts were collected in Eppendorf tubes and the left over PBS from the last washing was discarded so that only the cell pellet was left in each tube. For cell lysis, each time a freshly prepared lysis buffer was used with the following content: 500-1000 μL, depending on the number of samples, of cell lysis reagent, protease inhibitor cocktail, and both phosphatase inhibitor cocktails 1 and 2 at the final concentration of 1%, and 0.3 M of phenylmethylsulfonyl fluoride (PMSF). A lysis buffer 50 μL were added and samples were incubated for 30 min on ice with vigorous vortexing every 10 min, and final centrifugation for 10 min, at 13,000 rpm, and 4° C. The supernatants, representing the total cell lysate, were transferred to new Eppendorf tubes, and kept at −70° C., whereas the cell pellet were discarded. PIκBα-Kinetics According to H₂O₂-Stimulation. MCF7 breast cancer cells (2×10⁵) were seeded in 5 ml of RPMI 1640 medium using 25 ml plastic flasks and maintained at 37° C. After 24 h, the growth medium was substituted with a medium containing 0.5% FCS in order to minimize the activation of signal transduction cascades by the growth factors presented in the serum. Twenty-four hours later, cells were supplemented with 50 or 100 μM H₂O₂ for 5, 10, 20, 30, 40, and 60 min to detect inhibitory protein kappa B (IkBα) phosphorylation after H₂O₂ stimulation in MCF7 breast cancer cell lines.

As a control, no H₂O₂ treatment was used, according to which the kinetics of pIκBα were determined. Cells were collected, washed, and lysed as previously described. The experiments were performed in duplicates.

IKK Kinase Assay IκB kinase complex (IKK-β) activity was evaluated with IKK-β-inhibitor screening kit (Calbiochem, USA), an ELISA-based activity assay that utilizes a 50-amino acid GST-IkBαfusion polypeptide substrate that includes the Ser32 and Ser36 IKK-β phosphorylation sites. A hundred ng of the GST-IkBα substrate and 5 ng of recombinant IKK-β were incubated in the presence of 600 nM of the IKK-β inhibitor 5-(p-Fluorophenyl)-2-ureido]thiophene-3-carboxamide (Fuct), and 100 or 200 μg/ml of Coprinus comatus extracts. Reaction mixtures were added to wells precoated with glutathione to allow capture of GST-IkBα. The phosphorylated GST-IkBα substrate was detected using an anti-phospho-IκBα (Ser32/Ser36) antibody, followed by HRP-conjugation and color development with TMB substrate. The absorbance was monitored at 450 nm and is directly related to the level of IKK-β activity.

Densitometric Analysis. Densitometric quantitative analyses of the protein bands, detected by Western blot, have been carried out using the TotalLab software and are presented as folds of the volumes of protein bands. Densitometric analysis of the Western blots for pIκBα levels in response to the effects of the fungal extracts are presented as an average of the control values±SD.

4. Content and Chemical Composition of Coprinus comatus

Analysis of the polysaccharide composition of Coprinus comatus CBS 123401 submerged cultivated mycelial biomass (10 g) were washed with boiling 96% ethanol (300 ml) for 2 h. The insoluble residue (8.2 g, 82%) was extracted with water (300 ml) in autoclave at 120° C. twice for 1 h. A small portion of the extract was dried and analyzed by NMR, the rest dialyzed, concentrated to 100 ml, ultracentrifuged at 120000 g for 3 h, solution treated with a few drops of bromine for decolorization (some protein precipitated after this and was removed by centrifugation), separated by gel chromatography on Sephadex G-50 to give 3 fractions (150, 220, and 130 mg), containing starch, β-glucan and galactan in various proportions. The insoluble residue (5.8 g) was extracted with boiling 5% KOH for 3 h, solution dialyzed and treated as above, to give protein-β-glucan mixture (300 mg).

Methylation analysis. Samples (1-3 mg) were dissolved in 1 ml dry DMSO at 100° C. Insoluble glucans were left overnight, and still were not completely dissolved. After cooling to room temperature powdered NaOH (˜30 mg) was added, mixture stirred for 30 min, 0.5 ml of MeI was added, strirring continued for 30 min, excess MeI removed by air stream, water (5 ml) was added. For soluble samples the product was extracted by CH₂Cl₂, washed with water, hydrolyzed with 3 M TFA (120°, 3 h), reduced with NaBD₄, acetylated, analyzed by GC-MS. Insoluble compounds were recovered by dialysis and methylated one more time.

NMR experiments were carried out with a Varian INOVA 500 MHz spectrometer with a Varian Z gradient probe at 25° C. with acetone internal reference (2.225 ppm for ¹H and 31.5 ppm for ¹³C) using standard pulse sequences DQCOSY, TOCSY (mixing time 120 ms), NOESY (mixing time 200 ms), HSQC and HMBC (100 ms long range transfer delay). Data processing was done with Bruker Topspin program.

Monosaccharide analysis. Samples (1-5 mg) were dissolved in concentrated HCl (0.2 ml) at 40° C. for 1 h, inositol standard (0.5 mg) was added, mixture diluted with water (0.4 ml) and heated at 100° C. for 2 h. Acid was evaporated under air stream, sugars reduced with NaBH₄ (10 mg, 30 min), AcOH (0.5 ml) was added, samples dried and then dried twice from MeOH (1 ml), acetylated with 0.5 ml of Ac₂O, dried, analyzed by GC. This procedure gives improved recovery of glucose and glucosamine from insoluble polymers, but pentoses and 6-deoxyhexoses partly degrade. To get correct quantification of them, hydrolysis was performed with 3M TFA (120°, 3 h).

β-Glucan determination with Megazyme kit. General description: all glucans are solubilised in concentrated (37%; 10N) hydrochloric acid and then extensively hydrolysed by 1.3 N HCl at 100° C. for 2 h. Hydrolysis to D-glucose is completed by incubation with a mixture of highly purified exo-1,3-β-glucanase and β-glucosidase. The procedure was scaled down 10 times relative to the manufacturer instructions.

Measurement of total glucan (α-glucan+β-glucan) plus D-glucose in oligosaccharides, sucrose and free D-glucose. Glucans were extracted from samples (˜10 mg) with concentrated hydrochloric acid (0.2 ml) at 30° C. for 45 min with occasional vortex stirring. Acid was diluted 5 times with water and hydrolysis performed for 2 h at 100° C. Acid was neutralized with 1 ml of 2 M KOH, mixture diluted to 10 ml with 200 mM sodium acetate buffer (pH 5.0) and cleared by centrifugation at 1,500 g for 10 min. To 0.01 mL of the extract 0.1 ml of water and 0.1 ml of a mixture of exo-1,3-β-glucanase (20 U/mL) plus β-glucosidase (4 U/ml) in 200 mM sodium acetate buffer (pH 5.0) was added and mixture incubated at 40° C. for 60 min. 3.0 mL of glucose oxidase/peroxidase mixture (GOPOD) was added and incubated at 40° C. for 20 min. Absorbance at 510 nm was measured against the reagent blank.

Measurement of α-glucan (phytoglycogen and starch) plus glucose in sucrose and free d-glucose. Sample (10 mg) was extracted with 0.2 ml of 2 M KOH for 20 min with stirring. 0.8 mL of 1.2 M sodium acetate buffer (pH 3.8) was added followed by 0.02 mL of amyloglucosidase (1630 U/ml) plus invertase (500 U/mL) and incubated at 40° C. for 30 min with intermittent mixing. Samples diluted with water to 10 ml, cleared by centrifugation, 0.01 ml of sample was mixed with 0.01 ml of sodium acetate buffer (200 mM, pH 5.0) and 0.3 mL of GOPOD reagent and incubated at 40° C. for 20 min, absorbance at 510 nm was measured against the reagent blank. The reagent blank consists of 0.2 mL of sodium acetate buffer (200 mM, pH 5.0)+3.0 mL glucose oxidase/peroxidase reagent. The D-glucose standard consists of 0.1 mL D-glucose standard (1 mg/mL)+0.1 mL of sodium acetate buffer (200 mM, pH 5.0)+3.0 mL glucose oxidase/peroxidase reagent.

Extraction of Lectin Fraction from Submerged Cultivated Mycelium of Coprinus comatus CBS 123401.

Coprinus comatus mycelial biomass with a wet weight of 50 g were homogenized (⅙ w/v) under PBS (40 mM KH₂PO₄, 150 mM NaCl; pH 7.4) containing 1 mM phenylmethylsulfonyl fluoride. Homogenates were kept at 4° C. for 2 h and then centrifuged at 8000 g for 20 min. (NH₄)SO₄ at 80% saturation was used for precipitation of protein fraction. Mixtures were allowed to stand overnight at 4° C. The precipitates were collected by centrifugation at 12000 g for 20 min, dissolved in a minimal volume of PBS, and dialyzed successively in distillate water and then in PBS. The resulting extract was analyzed for protein quantity, lectin activity, and sugar specificity.

Assay for hemagglutinating activity of lectin. To measure the lectin hemagglutinating activity a trypsinized erythrocyte suspension was used. The rabbit blood was collected in a 150 mM tri-sodium citrate buffer containing 150 mM NaCl. An erythrocyte suspension was freshly prepared by washing the erythrocytes three times with ten volumes of washing buffer (PBS). Next, trypsinization of 4% erythrocytes suspension was carried out at 37° C. for 1 h; then erythrocytes were washed and suspended in the same buffer as 2% suspension (v/v).

In the assay for lectin (hemagglutinating) activity, a serial two-fold dilution of the lectin solution in microtiter U-plates (50 μl) was mixed with 50 μl of a 2% suspension of rabbit red blood cells in PBS (pH 7.4) at 20° C. The results were recorded after 1 h. The hemagglutination titer, defined as the reciprocal of the highest dilution exhibiting hemagglutination, was equal to one hemagglutination unit. Specific activity is the number of hemagglutination units per mg of protein.

Sugar-binding specificity of lectins. The investigation of inhibition of lectin-induced hemagglutination by various carbohydrates was performed in a manner analogous to the hemagglutination test. Sugar samples (0.3 M) were prepared in phosphate buffered saline. All of the dilutions were mixed with an equal volume (25 μL) of a solution of the lectin. The mixture was allowed to stand for 30 min at room temperature and then mixed with 50 μL of 2% rabbit erythrocyte suspension. Sugar-binding specificity was expressed as the minimum concentration of each sugar required for inhibition of hemagglutination of titer 4 of the lectin. N-Acetyl-D-galactosamine (GalNAc); N-Acetyl-D-glucosamine (GlcNAc), D(+)Galactose (Gal); D(+)Glucose, D(+)Lactose (Lac); D(+)Mannose, xylose, cellobiose, and dulcitol were tested for detection of sugar-binding specificity of lectins.

5. Characteristics of the Variety Tremella mesenterica CBS 123296; Submerged Cultivation of Single Cell Biomass in Erlenmeyer Flasks and Fermentor.

Preparation of cultures of Tremella mesenterica. Fruit bodies of Tremella mesenterica were collected in Israel on dead wood of Quercus sp. The basidiospore prints were obtained from a fresh fruit body situated under sterile Petri dish in a moist chamber with slowly decreasing humidity. Monosporous cultures were developed from basidiospore print spreading the spore suspension in sterile water onto the surface of malt agar in Petri dishes. Germinating basidiospores were investigated under stereomicroscope, and young colonies from yeast-like budding cells were transferred on to slants of malt agar (MA).

Inoculum of yeast-like haploid cells of Tremella mesenterica strains for the first step submerged culture was prepared as a suspension of 8-day old culture cells on MA slants in sterile water. A fermentation medium was inoculated by submerged culture, and a process for polysaccharide production was carried out in refrigerated orbital shaker at 220 rpm at a 27° C.

Polysaccharide production was estimated by alcohol precipitation of culture broth supernatant by 2 volumes of ethyl alcohol after separation cells of strain producer by centrifugation. A crude precipitate obtained from culture broth at the end of fermentation contains both extracellular polysaccharides and cells of strain producer.

A trace element mixture (micronutrients) composed of (g/l) 5.0 g/l FeSO₄.7H₂O; 0.625 g/l MnSO₄.H₂O; 0.435 g/l ZnSO₄.7H₂O and 0.2 g/l CuSO₄.5H₂O was prepared separately, and added to sterile culture media. The trace element mixture was added diluted 1:100 in culture medium to obtain a final cation concentration of (mg/l): Fe⁺⁺-10.0; Mn⁺⁺-2.0; Zn⁺⁺-1.0; Cu⁺⁺-0.5.

In order to prevent sediment formation of trace element hydroxides, pH of trace element solution is adjusted to 1.6-1.8 by 50% H₂SO₄ prior to sterilization by autoclaving.

Culture media of determined composition were developed from peptone and yeast extract (Pronadisa), and mineral salts (Sigma).

6. Content and Chemical Composition of Tremella mesenterica

Determination of acidic glucoronoxylomannan of Tremella mesenterica CBS 123296. Culture broth at the end of cultivation on fermentation medium was diluted three times in deionized water. Strain producer cells were separated by centrifugation for 10 min at 4° C. in an Eppendorf 5403 centrifuge at 5000 rpm. The supernatant was mixed with two volumes of ethyl alcohol, and crude polysaccharide was precipitated. The mixture of polysaccharides precipitated from culture broth was dissolved in water, and passed through a chromatography glass column filled with Amberlite IR-120 (H⁺-form). Acidic glucoronoxylomannan was precipitated from the collected fraction by gradual addition of 10% aqueous cetyl pyridinium chloride (CPC) until no more precipitate was formed. The insoluble CPC complex was collected by centrifugation, and dissolved into 10% sodium chloride. After separation of insoluble particles by centrifugation, the acidic polysaccharide was precipitated by addition of two volumes of ethanol.

The purified polysaccharide was obtained by dissolving the precipitate in water, followed by repeated precipitation with alcohol.

7. Interferonogenous and Immuno-Modulating Properties of Tremella mesenterica Preparations.

Determination of IFN level in blood plasma and functional activity of phagocytic cells The IFN level in the blood plasma was estimated by the oppression of the cytopathic influence of a test-virus (virus of vesicular stomatitis, Indiana strain—VSV) in the culture of mice fibroblasts cells L-929. Alveola of 96-alveola culture panel were filled with 100 μl of cell suspension (1×10⁶ cells/mL) and incubated for 18 hours at 37° C. in the atmosphere of 5% CO₂, then 100 μL of the double-diluted test samples were added. After 18 hours 50 μL of previously titrated work-dilute of VSV were added. The panels were incubated under the same conditions. The results were evaluated under microscope. The reciprocal of the final IFN dilution, which provided 50% cells-protection against cytopathic influence of the test-virus, was assumed as the IFN activity unit. The IFN titre was expressed in units/ml.

The biological activity of TNF in the blood plasma was evaluated by the cytolitic action on mice fibroblasts L-929. The indicator cells suspension (5×10⁶ cells/ml) was introduced in portions of 100 μl into flat-bottom 96-alveola-culture panel and cultivated for 24 hours at 37° C. in 5% CO₂ atmosphere. Then cultural medium was removed from the alveola and 100 μl of test sample and 100 μl of cultural medium containing actinomycin D (final concentration 1n/ml) were added to a mono-layer of cells. Cells cultivated without test samples served as a control. After 24 hours of incubation the cultural medium was removed, the cells were stained with 0.2% solution of crystal-violet during 10 min at a room temperature, washed out for three times with water and dried at 37° C.

The results were evaluated using Multi-scan DYNARECH (Swiss) at a wavelength of 550 nm.

The TNF activity was evaluated beyond the cytotoxicity index (CI), which was calculated using the following formula: CI=(K−D)/K×100%, Where K is the optical density in control alveola and D is the optical density in test alveola

TNF recombinant preparation “Rifnamen” made by “VECTOR” (Ukraine) was used for TNF testing.

Functional activity of phagocytosis system was investigated in the nitro blue tetrazolium recovery (NBT test) by cytochemical methods.

### 8. Tremella Extracts and Plant Resistance to Plant Viruses

Virus: Tobacco mosaic virus (TMV), strain U1;

Plants: Tobacco (Nicotiana tabacum L.) variety Immune 580 and Datura stramonium L., cultivated in a hothouse under conditions of natural lighting, humidity, and temperature. Plants at a stage of 4-6 leaves were used in the experiment.

Polysaccharides, extracted from culture liquid of Tremella mesenterica were employed in this study using the method of Kovalenko with some modifications. To isolate acid glucuronoxylomannan (GXM) from neutral polysaccharides, we applied its sedimentation from a water solution with acetone (1:1) in the presence of 0.05 M CaCl₂. Gas chromatography was carried out on Sephadex G-50 (2.5×80 cm) in pyridinium-acetate buffer, pH 4.5 (4 mL pyridine and 10 mL AcOH in 1 L water) and the eluate was monitored by refractive index detector.

In in vitro experiments the water solutions of the total preparation, neutral polysaccharide preparations, and pure GXM at concentrations of 10, 100, 500, and 1000 μg/mL were added to a suspension of TMV (10 μg/mL). After incubation over the 30 min the left halves of datura's leaves were inoculated with this mixture. The right halves of leaves were inoculated with TMV at the same concentration without polysaccharides. The degree of inhibition of viral infection, or VIR, was counted (in percentage) according to the number of local lesions on the experimental and control halves of the leaves by the following formula: I=((K−D)/K) 100%, where I—is a degree of virus inhibition or level of AVR, %; K—is the number (size) of local lesions in the control; D—is the number (size) of local lesions in the experiment.

Investigations of induced properties of GXM were carried out on the tobacco and datura plants. For this purpose, the hydrogen solutions of polysaccharides at concentrations of 1000-2500 μg/mL were injected into the intercellular space of the left halves of the leaves with a 1 mL (insulin) syringe. The two halves of leaves were inoculated by TMV (1-5 μg/mL). The degree of AVR was taken into account by the formula mentioned above.

When studying the GXM-induced mechanism of plant resistance, we used actinomycin D (AMD) inhibiting the DNA-dependent synthesis of mRNA by blocking RNA-polymerases. Solutions of AMD at the concentrations of 10 and 20 μg/mL and rhamnolipide (RL)−1 mg/mL, which were subepidermally introduced simultaneously or via definite periods of time after the GXM. Differences in the number and size of local lesions were assessed by the criterion of Student (t) test; the resulting α-value is displayed in the tables by the following symbols: +++p≦0.01%; ++0.1≦p≦1%; +1%≦p≦5%; 0p>5%.

### Example 1

### Characteristics of the variety Coprinus comatus CBS 123401

Vegetative mycelium in pure culture. Mycelial colony white, cottony, often develops “tufts” (hyphal aggregates) with maturity. Asymmetrically shaped, usually forms mycelial mats along the outer edge. Clamp connections, anastomoses, and hair-like crystals are often present on hypha.

Pileus 3-15 cm; oval to rounded-cylindrical when young, expanding to bell-shaped with a lifting margin; in age turning to black “ink”; dry; whitish with a brownish center; with large, shaggy scales; margin lined at maturity. Lamellae free from the stem; white, becoming pinkish, then black; turning to black “ink”; very crowded. Stipe 5-20 cm long; 1-2 cm thick; frequently tapering to apex; smooth; white; easily separable from cap; hollow, with a string-like strand of fibers hanging inside. Context white throughout, soft. Spore print black. Basidiospores 9-13×7-9 μm; elliptical; smooth; with a central to slightly eccentric pore. Basidia 4-spored, 28-43×10-13 μm, surrounded by 5-8 pseudoparaphyses. Pleurocystidia absent. Cheilocystidia variously shaped; up to 60×40 μm. Pileipellis made of cylindric elements 7-30μ wide. Only pseudoclamps presents.

Habitat. It grows in groups in places which are often unexpected, such as green areas in towns. It occurs widely in grasslands and meadows.

### Example 2

### Evaluation of Coprinus comatus Antioxidant Activity

2.1 Yield of Biomass and Extracts from Dried Submerged Cultivated Mushroom Mycelia

Nutrition medium selected for the submerged cultivation of higher Basidiomycetous mushrooms during the screening program ensured growth of all selected species. However, the yield of mycelial biomass after 8-11 days of Coprinus comatus CBS 123401 mushroom cultivation in identical culture conditions was 6.8 g/l (Table 1).

The extraction of soluble compounds from dried and milled mushroom mycelia was performed with culture liquid (instead of water) and ethanol. Significant differences in yield among the extracts received from different strains were revealed. Moreover, the yield of extracts received from mushroom biomasses significantly depended on the solvent used. The extraction with culture liquid of Coprinus comatus CBS 123401 biomass yielded 23.2% of extract.

Extraction with ethanol was less effective and a weak solubilizer used on the studied strain yielded only 8.5%.

2.2 Antioxidant Activity of Water (Culture Liquid) and Ethanol Extracts from Submerged Cultivated Coprinus comatus CBS 123401 Mushroom Mycelium

Data presented in Table 2 show the antioxidant activity (AOA) of water (Culture Liquid) extracts received from Coprinus comatus CBS 123401. Very high AOA was revealed when C. comatus CBS 123401 water extracts in concentration of 2 mg/mL were used. Slightly lower AOA was observed in water extracts from Coprinus comatus CBS 123401 mycelial biomas. The inhibition values of all these extracts practically did not change with an increase of their concentration in the reagent mixture.

The AOA of ethanol extracts from mushroom biomass not only depended largely on the higher Basidiomycetes species, but also on the variation of extract concentration in the reagent mixture. When the concentration of ethanol extract increased from 2 mg/ml to 4-8 mg/ml, the AOA of extracts from Coprinus comatus CBS 123401 increased from 74.4 to 86.4% (Table 3).

TABLE 2
Antioxidant activity (%) of water extracts from dried
submerged mushroom mycelia

| | Extract | | |
|---|---|---|---|
| | concentration | | |
| | (mg/mL) | | |
| Species | 2.0 | 4.0 | 8.0 |
| Coprinus comatus CBS 123401 | 83.8 | 88.4 | 91.6 |
| Standard BHA (mg/mL in methanol) | 98.2 | — | — |

BHA—Butylated hydroxyanisole

TABLE 3
Antioxidant activity (%) of ethanol extracts from dried
mushroom mycelia

| | Extract | | |
|---|---|---|---|
| | concentration | | |
| | (mg/ml) | | |
| Species | 2.0 | 4.0 | 8.0 |
| Coprinus comatus CBS 123401 | 74.4 | 81.3 | 86.4 |
| Standard BHA (mg/ml in methanol) | 98.2 | — | — |

BHA—butylated hydroxyanisole

2.3 Free-Radical Scavenging Activity of Water (Culture Liquid) and Ethanol Extracts from Submerged Cultivated Mushroom Mycelia

Free-radical scavenging is one of the known mechanisms by which antioxidants inhibit lipid oxidation. The method of scavenging 1,1-diphenyl picrilhidrazyl (DPPH) free radicals was used to evaluate the antioxidant activity. DPPH, a stable free radical generating substance with a characteristic absorption at 520 nm, was used to study the radical-scavenging effects of extracts. The decrease in absorbance is taken as a measure of the extent of radical-scavenging.

The highest free radical-scavenging activities of the water and ethanol extracts were measured at sample concentrations of 0.5 mg/ml (27.0% and 22.0%, respectively), but the values were much lower than that of the standard (92.0%) (Tables 4 and 5).

The free radical-scavenging capacities of ethanol extracts of Coprinus comatus CBS 123401 decreased from 22 to 1, with a sample concentration increase from 0.5 to 9 mg/ml %, respectively. (Table 5).

TABLE 4
Scavenging ability (% of inhibition) and EC50 values of water
(culture liquid) extracts from the submerged mushroom mycelium
of Coprinus comatus CBS 123401

| | Water extract (mg/ml) | | | | EC₅₀ |
|---|---|---|---|---|---|
| Species | 0.5 | 1.5 | 3.0 | 9.0 | (mg/ml) |
| Coprinus comatus | 27 ± 2.7 | 22 ± 4.2 | 15 ± 2.1 | 0 | 1.7 ± 0.3 |
| 123401 | | | | | |

| | |
|---|---|
| BHA (1 mg/ml) | 92 ± 2.1 |

BHA—Butylated hydroxyanisole;
EC₅₀—Half maximal effective concentration

The values of effective concentrations for DPPH scavenging effects given in Tables 5 and 6 show evidence that the EC₅₀ of both water and ethanol extracts appeared to be near 1 mg/mL.

TABLE 5
Scavenging ability (% of inhibition) and EC₅₀ values of ethanol
extracts from the submerged mushroom mycelium of
Coprinus comatus CBS 123401

| | Ethanol extract (mg/ml) | | | | EC₅₀ |
|---|---|---|---|---|---|
| Species | 0.5 | 1.5 | 3.0 | 9.0 | (mg/ml) |
| C. comatus | 22 ± 2.1 | 17 ± 1.7 | 5 ± 1.4 | 3 ± 0.4 | 1.3 ± 0.4 |
| CBS 123401 | | | | | |
| BHA | | | 92 ± 2.1 | | |
| (1 mg/ml) | | | | | |

BHA—Butylated hydroxyanisole;
EC₅₀—Half maximal effective concentration

### Example 3

### Study on Biological Activity of Water (Culture Liquid) and Ethyl Acetate Extracts from Submerged Cultivated Mycelium of Coprinus comatus CBS 123401

3.1 pIκBα Levels after NF-κB Stimulation in MCF7 Cells by H₂O₂

Numerous studies on medicinal mushrooms proved their exclusive potential not only as dietary supplements and immunoenhancers, but also as the source of modulators of various cellular responses. Despite the observed success of most of the chemotherapeutic regimes, cellular adaptations have enabled tumor cells to become resistant to many chemotherapeutic drugs. One of these cellular chemo-resistance factors is the nuclear factor kappa B (NF-κB), which was shown to promote tumor proliferation. The activity of NF-κB is tightly regulated by interaction with IκB proteins. As with the NF-κB proteins, there are several IκB proteins that have different affinities for individual NF-κB complexes, but are regulated slightly differently and expressed in a tissue-specific manner.

Extracellular stimuli lead to the activation of IκB kinase (IKK) that phosphorylates IκB on two conserved serines (Ser32 and Ser36 in IkBα). This phosphorylation marks IκB for proteasomal degradation, resulting in the nuclear translocation and activation of NF-κB. This is considered the classic pathway of NF-κB activation. Several alternative pathways of NF-κB activation have been described. In the current study of the activation of NF-κB, hydrogen peroxide was used, which is a well known NF-κB activator and a strong oxidative reagent. The preliminary data showed that a 10-min treatment with 100 μM of H₂O₂ caused the highest level of pIkBα, and treatment with 10 μM of curcumin, which is a known antioxidant and anticancer reagent, significantly inhibited pIκBα level. MCF7 cells were stimulated with 50 and 100 μM H₂O₂ for increasing time periods. For 20 and 40 min 50 μM of H₂O₂ stimulation showed high level of phκBα compared to the control, DMSO (dimethyl sulfoxide)-treated cells, where no H₂O₂ was added. The highest level of pIκBα was shown by 100 μM H₂O₂-treated cells at 10-min stimulation (not shown. Therefore, the best conditions for checking the potential effects of extracts on pIkBα levels were established at 10 min H₂O₂-stimulation along with selected fungal extracts.

(MCF7 cells were stimulated with 50 and 100 μM of H₂O₂ for increasing time periods. Stimulation with 100 μM of H₂O₂ at 10 min intervals showed the highest level of pIκBα compared to the control, using DMSO-treated cells where no H₂O₂ was added. Data represent results of one of two similar experiments).

3.2 Effect of Coprinus comatus CBS 123401 Crude Extracts on Cell Viability

In the current experiments, Coprinus comatus CBS 123401 was investigated. C. comatus cultural liquid (water) (E1) and ethyl acetate (E2) crude extracts were tested for their ability to affect cell viability of MCF7 cell line and IC₅₀s were calculated. Extract E2 appeared to be the most potent with IC₅₀ of 32 μg/ml, followed by E1 with IC₅₀ of 76 μg/ml (Table 6). These results indicate that the active moieties of C. comatus CBS 123401 extract were concentrated in extract E2. Extract E1 did not affect cell viability in a significant manner, however, this assay revealed that extract E2 demonstrated higher growth inhibition activity than E1, arguing that E2 might possess some bioactive compounds with possible anticancer activity.

TABLE 6
Cell cytotoxicity of Coprinus comatus CBS 123401 extracts

| Coprinus comatus | |
|---|---|
| CBS 123401 crude | IC₅₀ ± SD μg/ml |
| E1 (Water extract) | 76 ± 1.41 |
| E2 (Ethyl acetate extract) | 32 ± 0.71 |

Cells were treated with increasing concentrations of Coprinus comatus extracts. After 48 h, cells were collected, stained with 0.4% trypan blue solution (1:1), and counted using a hemacytometer. Percent inhibition of cells' viability was calculated against the DMSO-treated control. Values (μg/ml) are represented as the mean IC₅₀s of a duplicated experiment±standard deviation.

3.3 Coprinus comatus CBS 123401 Crude Extracts Effect on the IκBα Phosphorylation

The NF-κB pathway has emerged as one of the most promising targets in cancer drug discovery. Coprinus comatus CBS 123401 extracts were found to modulate the NF-κB activation pathway. In order to establish the effects of the two extracts of C. comatus, E1 and E2, on pIkBα level, MCF7 cells were stimulated for 10 min with 100 μM of H₂O₂ as described above. The preliminary data showed that a 10-min treatment with 100 μM H₂O₂ caused the highest level of pIκBα (not shown). C. comatus extracts were tested for their ability to affect κBα phosphorylation in 10-min cell stimulation with H₂O₂. Results showed that both extracts significantly affected IκBαphosphorylation in a dose-dependent manner. The organic extract E2 appeared to be the most active inhibitor of IκBα phosphorylation in both concentrations (100 and 200 μg/mL) used. As presented in FIG. 2, extract E2 almost completely inhibited IκBα phosphorylation at the concentration 200 μg/mL and caused partial inhibition in the phospho-IκBα level at a concentration of 100 μg/mL. The extracts' effect is comparable to the effect of curcumin, which also demonstrated a high phospho-IkBα inhibitory effect (FIG. 2).

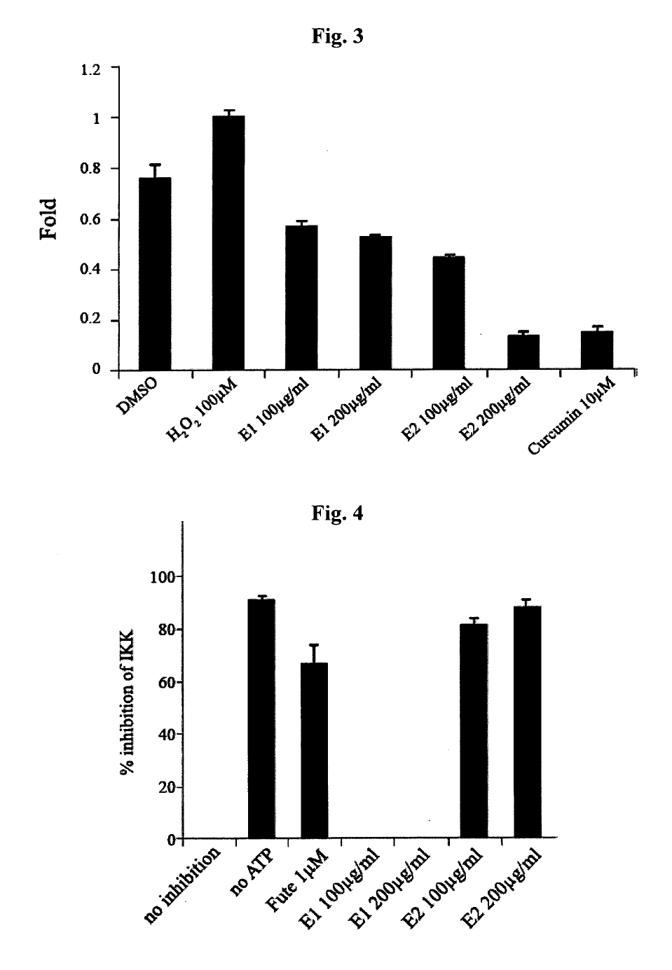

In contrast with these results, the extract E1 at concentrations of 100 and 200 μg/ml only partially inhibited the phosphorylation of IkBα(FIG. 3).

The fact that E2 extract was a much more potent inhibitor than E1 extract indicates that lipid soluble substances are quite potent inhibitors of the NF-κB pathway.

3.4 The Effect of Coprinus comatus E1 and E2 Extracts on the IKK Activity

The major activator of NF-κB is the IkB kinase complex (IKK). This complex includes two catalytic subunits, IKK-α and IKK-β, and a regulatory subunit, IKK-c (also known as NEMO). The NF-κB pathway is triggered by bacterial and viral infections as well as pro-inflammatory cytokines and chemokines (e.g., tumor necrosis factor a (TNF-a), lipopolysaccharide (LPS), interleukins (IL-1, IL-6), etc.), all of which activate the IKK complex phosphorylation by IKK β of two specific serines near the N terminus of IκBα, which targets IκBα for ubiquitination and degradation by the proteasome. Almost all signals that lead to the activation of NF-κB converge on the activation of a high-molecular-weight complex that contains a serine-specific IKK. Activation of the IKK complex leads to phosphorylation by IKK β of two specific serines near the N terminus of IκBα, which targets IκBα for ubiquitination and degradation by the proteasome.

On the basis of the abovementioned results it was assumed that the NF-κB inhibitory effects of the active extracts are due to modulation of the NF-κB pathway upstream of IkBαphosphorylation. IKK complex phosphorylates IκBα at serines 32 and 36, which leads to ubiquitination and degradation of IκBα by the 26S proteasomes. In order to establish whether E1 and E2 affect IKK activity, an ELISA-based IKK activity assay was applied. The effect of E1 and E2 extracts on IKK activity is presented in FIG. 4. Received data showed that only the E2 extract inhibited the activity of IKK complex as compared to the control of the untreated sample. Moreover, E2 exhibited a strong effect, comparable to the effect of the positive controls used, 5-(p-Fluorophenyl)-2-ureido]thiophene-3 carboxamide (Fuct), and to MO04-Marasmius oreades crude ethyl acetate extract.

### Example 4

### Content and Chemical Composition of Submerged Cultivated Mycelium of Coprinus comatus CBS 123401

The chemical composition of submerged cultivated mycelium of Coprinus comatus was established by conventional methods well known in the art and is disclosed in Table 7 and Table 8.

### 4.1 Chemical Content.

TABLE 7
Content and chemical composition of Coprinus comatus CBS 123401

| Test | Units | Result | Comment |
|---|---|---|---|
| Energy | Kcal/100 gr | 349 | 1 |
| Protein | % | 37 | 2 |
| Water content | % | 13.5 | — |
| Ash | % | 5.5 | — |
| Fats and Oils | % | 5 | — |
| Carbohydrates | % | 39 | 3 |
| Profile of the fatty acid GC-FID | | | |
| Pentadecanoic acid (C15:0) | % | 1.4 | |
| palmitic (C16:0) | % | 11.8 | |
| palmitoleic acid (C16:1) | % | 5.4 | |
| heptadecanoic acid (C17.0) | % | 0.8 | |
| stearic (C18:0) | % | 3.3 | |
| Oleic (C18:1n9c) | % | 6.1 | |
| Linoleic (C18:2n6t) | % | 0.4 | |
| Linoleic (C18:2n6c) | % | 66.6 | |
| Linolenic (C18:3n3) | % | 1.1 | |
| Linolenic acid (C18:3n6-γ) | % | 1.1 | |
| Arachidic acid (C20:0) | % | 0.2 | |
| Heneicosanoic acid (C21:0) | % | 0.3 | |
| Behenic acid (C22:0) | % | 0.5 | |
| Lignoceric acid (24:00) | % | 0.6 | |
| Full metal screening at ICP | | | |
| Al—Aluminium | mg/Kg | <10 | |
| As—Arsenic | mg/Kg | 0.2 | |
| B—Boron | mg/Kg | 4 | |
| Ba—Barium | mg/Kg | 0.4 | |
| Be—Beryllium | mg/Kg | <0.5 | |
| Ca—Calcium | mg/Kg | 4047 | |
| Cd—Cadmium | mg/Kg | <0.1 | |
| Co—Cobalt | mg/Kg | 0.2 | |
| Cr—Chromium | mg/Kg | 0.3 | |
| Cu—Copper | mg/Kg | 8 | |
| Fe—Iron | mg/Kg | 41 | |
| Hg—Mercury | mg/Kg | <0.5 | |
| K—Potassium | mg/Kg | 14525 | |
| Li—Lithium | mg/Kg | <0.5 | |
| Mg—Magnesium | mg/Kg | 2124 | |
| Mn—Manganese | mg/Kg | 2 | |
| Mo—Molybdenum | mg/Kg | <0.5 | |
| Na—Sodium | mg/Kg | 1136 | |
| Ni—Nickel | mg/Kg | 1 | |
| P—Phosphorus | mg/Kg | 11359 | |
| Pb—Lead | mg/Kg | 0.3 | |
| S—Sulfur | mg/Kg | 3596 | |
| Se—Selenium | mg/Kg | 0.6 | |
| Sn—Tin | mg/Kg | 0.8 | |
| Sr—Strontium | mg/Kg | 2 | |
| Ti—Titanium | mg/Kg | 1 | |
| V—Vanadium | mg/Kg | 0.3 | |
| Zn—Zinc | mg/Kg | 74 | |

— = No comments
1. Energy—calculated from the content of protein, fats and carbohydrates at the sample.
2. Protein—protein calculation factor 6.25.
3. Carbohydrates—the result was calculated according to content of: water, ash, fat and protein at the sample.
4. Test results are given at relative percentage from the oil.
5. Metal screening—result with ‘<’—trails are not found at the mentioned sensitivity limit.

Mycelia of Coprinus comatus CBS 123401 contained 17 free amino acids, 10 of which are essential amino acids (with asterics): γ-aminobutyric acid*, alanine, arginine, aspartic acid, glutaminic acid, glycine, histidine*, isoleicine*, leucine*, lysine*, methionine*, phenylalanine*, serine, threonine*, tryptophan*, tyrosine, and valine*.

TABLE 8
Vitamin content of Coprinus comatus CBS 123401

| Test | Units | Result | Comment |
|---|---|---|---|
| Vitamin B1 (Thiamine) | mg/100 gr | 7.5 | |
| Vitamin B2 (Riboflavin) | mg/100 gr | 0.45 | |
| Vitamin A (Retinol) | μg/100 gr | <50 | |
| Vitamin C (Ascorbic acid) | mg/100 gr | 6.15 | |
| Vitamin E (α-Tocopherol) | mg/100 gr | <1 | |
| Vitamin B₃ (Niacin + N.amid) | mg/100 gr | <1 | |

4.2 Analysis of the Polysaccharide Composition of Coprinus comatus CBS 123401 Submerged Cultivated Biomass.

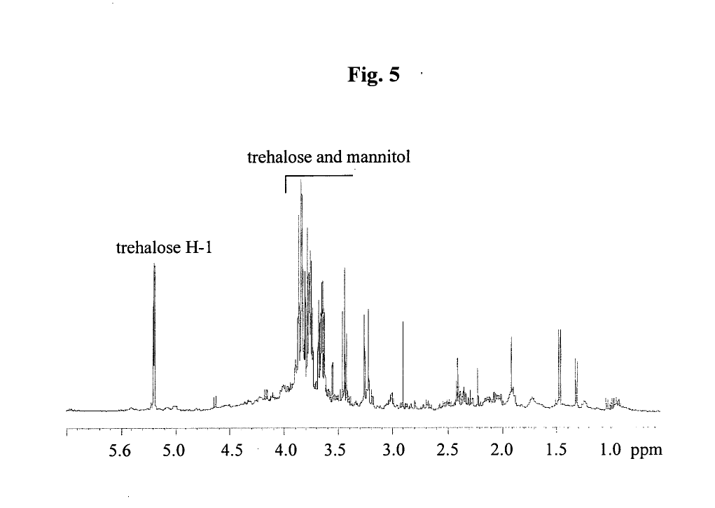

Washed cells were extracted by hot water in an autoclave at 120° C. for 1 h; the precipitate was removed by centrifugation. A small amount of solution was dried and analyzed by NMR. The spectra (FIG. 5) showed intense sharp signals of low-molecular mass components and typical protein-polysaccharide background. 2D spectra of this product were recorded and low-molecular mass components were identified as trehalose (α-Glc-1-1-α-Glc, a characteristic component of mushrooms such as shiitake (Lentinus edodes), maitake (Grifola fondosa), nameko (Pholiota nameko), and Judas's ear (Auricularia auricula-judae), which can contain 1% to 17% percent of trehalose in dry weight form), and mannitol, in the molar ratio ˜1:1. Mannitol content was estimated by GC analysis: 1% w/w of inositol (internal standard) was added to the fresh cells, and the mixture was acetylated by acetic anhydride-pyridine at 100° C. for 1 h. GC analysis showed that the mannitol content of the cells was 3% by weight, consequently trehalose content is 6% (it has molecular mass twice that of the mannitol).

The residue after water extraction was treated with 5% NaOH (100° C., 3 h); precipitate was removed by centrifugation; solution was dialyzed and dried; the yield was 400 mg; protein content was 30% and carbohydrate content was 55%.

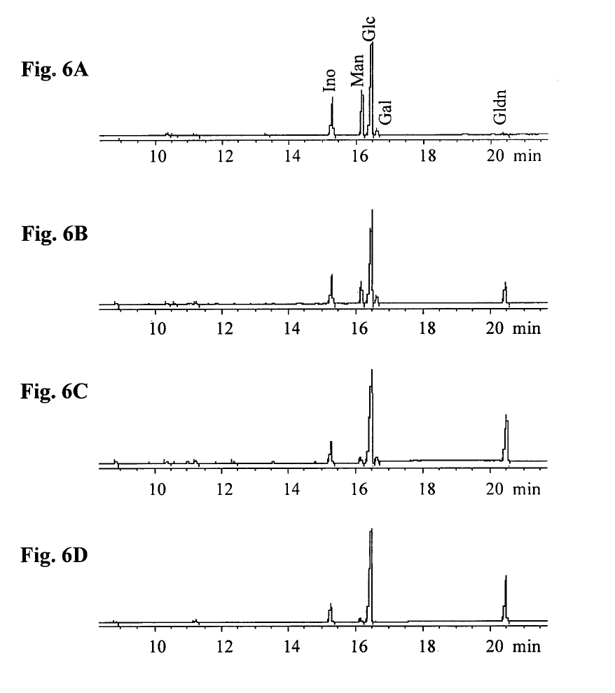

Monosaccharide analysis of the whole cells, extracts, and residues after extraction was performed; results are shown in the Table 9 and FIG. 6. Cells and solid residues were dissolved in concentrated HCl at 40° C. for 1 h and then diluted with water to a final acid concentration of 2M. Other samples were directly dissolved in 2M HCl. Hydrolysis was carried for 2 h at 100° C., products were dried, sugars were converted into alditol acetates (NaBH₄ reduction and acetylation with Ac₂O), and analyzed by GC. Glucose content of the solid residues after extraction increases because insoluble β-glucan remains in the residue, and soluble components are progressively removed by extraction.

TABLE 9
Monosaccharide composition of Coprinus comatus and extracts

| | Man | Glc | Gal | GlcN |
|---|---|---|---|---|
| EtOH washed cells | 6 | 48 | 2 | 10 |
| Water extract | 8 | 26 | 3 | 0 |
| NaOH extract | 2 | 60 | 2 | 0 |
| Cells without hydrolysis | 3 | | | |
| Cells after water extraction | 2 | 40 | 2 | 25 |
| Cells after water and NaOH | 1 | 30 | 0 | 50 |

Monosaccharide composition of the cells and extracts (% w/w), measured by GC relative to internal inositol.
Samples were dissolved in concentrated HCl (37°, 1 h), diluted with 5 vol of water to 2M HCl, heated 2 h at 100°, reduced and acetylated.
“Cells without hydrolysis” were just acetylated to measure the content of free mannitol

Removal of low-molecular mass components from the water extract by dialysis left a product, containing 25% carbohydrates and 40% proteins as determined by colorimetric tests (phenol-sulfuric acid for the polysaccharides and Lowry for proteins). Polysaccharides were identified as starch, β-glucan, and fucogalactan on the basis of NMR spectra. Starch amylose (2% w/w from cells) has been isolated in pure form by CaCl₂ precipitation. Galactan content was estimated as 3% from the monosaccharide analysis. Fucose content of the whole extract and cells was too low for quantification. The structure of the galactan was analyzed by NMR and methylation and is shown below:

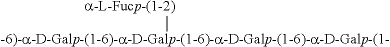

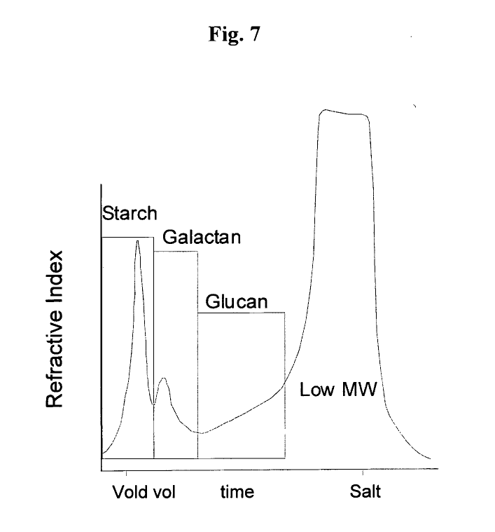

Polysaccharides from water and NaOH extracts were partially separated by size-exclusion chromatography on Sephadex G-50 (FIG. 7). Starch was eluted first and was identified by ¹H NMR spectrum. Galactan also was eluted close to the void volume, which agreed with published data on its molecular mass, estimated as 10,000 Da. Void volume (excluded volume) for Sephadex G-50 is 10,000 Da for dextran. Galactan was eluted close to ovoid, but slightly retained (thus its molecular mass was not greater than excluded mass), so it had max mass of 10,000 Da. It also was presented in lower mass fractions.

Soluble β-glucan had wide distribution of molecular mass from 10,000 to <1000 with no visible dominant mass. As one can see from the chromatogram (FIG. 7) β-glucan was elited as a very broad peak from vpid volume to the salts, spanning whole fractionation range of Sephafex G-50 (500-10,000 Da). It was not possible to prepare pure β-glucan; it contained some amount of starch, galactan, and protein. Anion-exchange chromatography improved spectra quality due to the removal of part of protein, but polysaccharides did not separate completely. Because of low-molecular mass of glucan, some amount of glucan was lost during dialysis and gel chromatography.

Methylation analysis of (3-glucan showed the presence of terminal, 3-, 4-, 6-, and 3,6-substituted glucose residues in the ratio of 2:1:0.3:3:1. The methylation analyses suggests that side chains are attached to every third glucose of the main chain and that the average length of the side chain is 3 sugars (t-Glc:6-Glc˜1:2, 3-Glc:3,6-Glc˜2:1). The presence of 4- and 4,6-substituted Glc may originate from starch. Peaks of 2- and 2,6-substituted Gal belonged to the galactan.

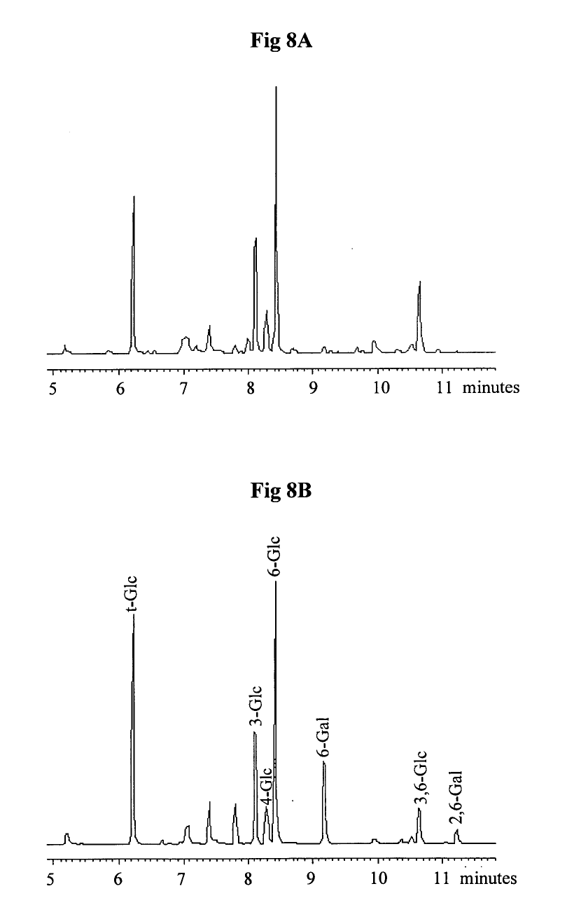

NMR analysis of the β-glucan fraction showed its similarity to Ganoderma glucan (FIG. 8 and Table 10). The structure is shown below:

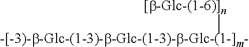

wherein m is an integer between 1 and about 10 and n is an integer equal to about 3.

For the quantification of the starch and insoluble β-glucan content the Megazyme assay “Mushroom and beta-glucan” was used. Measurements were performed according to the manufacturer's instructions. Samples of cells, water-extracted cells, water and NaOH-extracted cells, water extract and NaOH extract were analyzed. The results are shown in Table 11. Megazyme assay “Mushroom and beta-glucan” is based on the measurement of total glucose, released by a combination of hydrolysis and enzymatic treatment and a separate measurement of the starch content. β-glucan content is calculated as the difference of the first two numbers. No direct measurement of β-glucan is used.

TABLE 10
NMR data for the Coprinus comatus β-glucan (ppm; D2O, 25° C.).

| | | H/ | H/ | H/ | H/ | H/ | H/ |
|---|---|---|---|---|---|---|---|
| Unit | Nucleus | C-1 | C-2 | C-3 | C-4 | C-5 | C-6 |
| Glc | H | 4.76 | 3.31 | 3.51 | 3.40 | 3.46 | 3.72/3.91 |
| | C | 104.2 | 74.7 | 77.1 | 71.1 | 77.4 | 62.3 |
| −3-Glc | H | 4.52 | 3.51 | 3.75 | 3.51 | 3.50 | 3.72/3.91 |
| | C | 104.2 | 74.5 | 85.9 | 69.7 | 77.1 | 62.3 |
| −3,6-Glc | H | 4.52 | 3.51 | 3.75 | 3.57 | 3.62 | 3.86/4.20 |
| | C | 104.2 | 74.5 | 85.9 | 69.6 | 76.4 | 70.4 |

TABLE 11
Total glucan, α-glucan and β-glucan content of
Coprinus comatus samples

| | Glucan content % w/w | | |
|---|---|---|---|
| | Total | α- | β-Glucan |
| EtOH washed cells | 46.6 | 5.1 | 41.5 |
| Water extract | 28.2 | 15.1 | 13.1 |
| NaOH extract | 35.3 | 12.3 | 22.0 |
| Cells after water extraction | 42.0 | 8.0 | 34.0 |
| Cells after water and NaOH | 35 | 5 | 30 |

### CONCLUSION

The results of this study show that Coprinus comatus produces β-glucan with β-1-3-glucose main chain with 1-6 linked side chains, similar to the soluble β-glucan extracted from Ganoderma lucidum mushrooms. C. comatus also produces fucogalactan, starch, trehalose and mannitol; all of these components were previously found in mushrooms. Soluble β-glucan has low-molecular mass and is easily extractable with hot water.

4.3 Lectin content. Tested species Corpinus comatus CBS 123401 biomass possesses hemagglutinating activity (Table 12). A very high lectin hemagglutinating titer (18585) was revealed in biomass extracts from C. comatus. Specific hemagglutinating activity of C. comatus reached 62700 U mg⁻¹.

TABLE 12
Corpinus comatus CBS 123401 biomass protein content,
hemagglutinating activity and sugars specificity

| | Total | HA | Specific HA | Specificity | MIC of |
|---|---|---|---|---|---|
| | protein | titer, | activity | of | saccharides |
| Species | (mg) | (T-1) | (U mg−1) | saccharides | (mM) |
| Corpinus | 71.5 | 18585 | 62700 | Lac, | Lac (0.80) |
| comatus | | | | GlcNAc, | |
| | | | | Gal | |

MIC = minimal inhibition concentrations
HA = hemagglutinating activity

The data show that lactose followed by GalNAc and galactose are the most widespread inhibitors of hemagglutinating activity of tested mushroom lectin. Lactose was the most potent inhibitor of fungi. Lactose inhibited lectin activity of C. comatus at a concentration of 0.78 mM. At the same time, xylose, cellobiose, and dulcitol did not inhibit the lectin hemagglutinating activity of Coprinus comatus biomass

Obtained result showed that C. comatus have the capability to accumulate lectin in biomass and fruit bodies. Especially high hemagglutinating activity was revealed in C. comatus. The determination of sugar specificity toward mushroom lectins showed that lactose binding lectins are the most widespread among the tested strain.

### Example 5

### Characteristics of the Variety Tremella mesenterica CBS 123296

Vegetative mycelium in pure culture. Mycelial colony white, cottony, often develops “tufts” (hyphal aggregates) with maturity. Asymmetrically shaped usually forms mycelial mats along the outer edge. Clamp connections, anastomoses, and hair-like crystals are often present on hypha.

Basidiocarps often large and conspicuous, 2-10 cm wide and up to 5 cm high, mostly solitary, gelatinous, cerebriform when young and moist, later foliose with irregular clustered folds, consisting of several undulate-plicate lobes, yellow to yellowish-orange in fresh specimens, occasionally paler or entirely unpigmented and white when old, in wet or dark environments, yellowish-orange to darker when dry. Hymenial surface smooth, more or less shiny. Flesh gelatinous and soft. Hyphal system monomitic. Hyphae with abundant clamp connections, hyaline, thin- to thick-walled, mostly 1.5-3 μm wide, somewhat wider in the inner part of the basidiocarp, gelatinized. Haustorial cells globose to oblong, 3-6×2-4 μm wide, usually present except in young specimens, mostly abundant close to attachment point. Basidia of two types: 1) broadly ellipsoid to subglobose, 10-25×10-22 μm; 2) oval to clavate, 20-30(-35)×12-20 μm; longitudinally or obliquely septate, four spored, with sterigmata 100-150×2-3 μm wide, apically swollen up to 7 μm. Basidiospores broadly ellipsoid to oblong, (8-)10-16(-18)×(5-)7-10(-12) μm, smooth, hyaline, thin-walled, with an evident apiculus, negative in Melzer's reagent. Conidiophores densely branched, often abundant in the hymenium, particularly present in young specimens. Conidia subglobose to oval and, 3-5×2.5-3.5 μm, or ellipsoid to cylindrical 3-5×(1-)2 μm in diam., smooth, hyaline, often numerous. Hyphidia usually absent, sometimes present in early stages of development, thin to slightly thick-walled, up to 3.5(-4) μm wide. Vesicles variable in shape and size, ranging from globose to ellipsoid, mostly 20-30×5-10 μm, thick-walled. Spore print whitish or pale yellowish.

Habitat: on hardwood decayed branches, logs, and sticks.

Tremella mesenterica CBS 123296 has a special life cycle. In contrast to other higher Basidiomycetes mushrooms a single basidiospore germinates on a nutrient medium broth by hypha and by yeast-like budding cells. Monobasidiosporous culture is haploid, i.e. contains only one nucleus in each cell. When two compatible haploid cells, originating from different basidiospores, come into contact, a plasmogamy and caryogamy occurs and dycariotic mycelium develops. The dycariotic mycelium cannot grow in the form of budding cells, under any conditions of cultivation, so a yeast-like type of growth is genetically determined by a haploid status of mushroom culture. The haploid culture is more plastic, because on poor media or under conditions of exhaustion yeast-like cells can form haploid hypha. One-cell fungi cultures, like other microorganisms, are more acceptable for biotechnological processes, than mycelial ones. This is especially important for Basidiomycetes dycariotic cultures, which grow in the form of sterile mycelium, and a special procedure for preparing Basidiomycetes inoculum is needed, that includes dycariotic mycelium homogenization. The haploid yeast-like budding culture of the present invention, is the optimal form of growth not only from biotechnological considerations, but as defined by its physiological attribute of producing a larger amount of polysaccharide than mycelium form.

### Example 6

### Regulation of Growth and Biosynthetic Activity of Medicinal Jelly Mushroom Tremella mesenterica CBS 123296 Pure Culture

Fruit bodies of Tremella mesenterica were collected for basidiospore print development. Fruit bodies were associated with Peniophora sp. on a dead twig of Quercus sp. Haploid yeast-like cultures obtained from this specimen demonstrated fast growth on agar media, and 14 of them were used for primary screening in submerged culture conditions. A strain selected is deposited at Haifa University Culture Collection (HAI) under the name Tremella mesenterica CBS 123296.

Optimum pH value for selected strain biomass growth in submerged culture conditions was determined in a range from 5.5 to neutral meaning. The highest yield of biomass was obtained on liquid malt extract medium with pH 6.35.

Optimization of culture medium composition suitable for biomass accumulation have demonstrated that sucrose is as appropriate a source of carbon as is glucose.

A mixture of peptone and yeast extract is a good source of nitrogen, while ammonia salts investigated previously decreased pH of a media very rapidly. They contained also enough phosphorus for cell growth, as addition of phosphorus salts did not resulted in increased yield of biomass. Magnesium acetate is considered as more physiologically alkaline than MgSO₄, and indeed prevented rapid acidifying of culture media.

Slightly modified culture medium was used further as a standard “inoculum media” of a following composition (g/l): sucrose—20.0; peptone—2.0; yeast extract—2.25; Mg acetate—1.0; KCl—1.0. pH of the medium after sterilization at 120° C. for 30 min. is close to 6.5.

The optimal composition of the “fermentation medium” was found to be as follows: Tremella mesenterica was cultivated in the medium prepared by mixing Part A consisting of (g/L) Sucrose, 50.0; Yeast extract 0.5; KCl, 1.0; Mg acetate.4H₂O, 1.0 with Part B consisting of (g/L) NaH₂PO₄.H₂O, 0.5 and Na₂HPO₄.7H₂O, 1.0.

Part B was added to a flask with part A when solutions were cold to room temperature after sterilization.

Cultivation of T. mesenterica was continued for 72 h at which point TMP in culture liquid reached 27.0 g/l (Table 13). After precipitation, separation and drying 170 g of TMP preparation was received.

TABLE 13
Culture parameters change during Tremella mesenterica CBS 123296
cultivation in fermentor.

| | Medium | | Agitation, | |
|---|---|---|---|---|
| Time, h | pH | DO, % | rpm | TMP, g/L |
| 0 | 6.35 | 80.7 | 50 | 1.8 |
| 2 | 6.29 | 50.3 | 50 | |
| 5 | 6.12 | 31.4 | 50 | |
| 11 | 5.80 | 19.4 | 184 | |
| 23 | 3.26 | 11.8 | 302 | |
| 24 | 5.00 | 14.3 | 304 | 7.5 |
| 33 | 5.00 | 19.3 | 380 | |
| 36 | 5.00 | 8.2 | 400 | |
| 48 | 5.00 | 1.7 | 400 | 22.3 |
| 72 | 5.00 | 2.3 | 400 | 27.0 |

DO, Dissolved oxygen;
TMP, crude T. mesenterica preparation

### Example 7

### Content and Chemical Composition of Tremella mesenterica

7.1 General composition. Tremella mesenterica CBS 123296 submerged culture crude product consists of cell biomass of a strain-producer and exocellular polysaccharide glucoronoxylomannan in equal proportion. The general composition in this case is presented in Table 14.

TABLE 14
General composition of Tremella mesenterica CBS 123296

| Test | % |
|---|---|
| Acidic α-(1-3) heteropolysaccharide | 35.0 |
| glucoronoxylomannan | |
| Glycogen | 6.0 |
| Crude Dietary Fiber & B-glucan | 31.8 |
| Mineral Elements | 6.2 |
| Protein | 20.0 |
| Free amino acids | 1.0 |

The chemical composition was determined at two different and independent facilities in Israel (Bactchem Co. and Aminolabs Co.), and is disclosed in Tables 15 to 17.

TABLE 15
Chemical composition of submerged biomass Tremella
mesenterica CBS

| Type of analysis | Units | Results |
|---|---|---|
| Oleic acid-C18:1 | gr/100 g | 1.6835 |
| Linoleic acid-C18:2 | gr/100 g | 1.1618 |
| Palmic acid-C16:0 | gr/100 g | 0.725 |
| Palmitoleic acid-C16:1 | gr/100 g | 0.0185 |
| Stearic acid-C18:0 | gr/100 g | 0.0888 |
| Myristic acid | gr/100 g | 0.015 |

Microelements

| | | |
|---|---|---|
| Ag—Silver (in ICP) | mg/100 g | <0.2 |
| Al—Aluminum (in ICP) | mg/100 g | 0.7 |
| Ac—Arsenic (in ICP) | mg/100 g | <0.050 |
| B—Boron (in ICP) | mg/100 g | <0.24 |
| Ba—Barium (in ICP) | mg/100 g | 0.3 |
| Be—Beryllium (in ICP) | mg/100 g | <0.005 |
| Ca—Calcium (in ICP) | mg/100 g | 11766 |
| Cd—Cadmium (in ICP) | mg/100 g | <0.050 |
| Co—Cobalt (in ICP) | mg/100 g | <0.010 |
| Cr—Chromium (in ICP) | mg/100 g | 0.16 |
| Cu—Copper (in ICP) | mg/100 g | 0.26 |
| Fe—Iron (in ICP) | mg/100 g | 1.5 |
| Hg—Mercury (in ICP) | mg/100 g | <0.025 |

TABLE 16
Amino acid composition of submerged biomass of
Tremella mesenterica CBS 123296

| Sample | | Submerged | |
|---|---|---|---|
| Analysis | Units | mycelium | |
| Cysteic acid | % | 0.02 | 2 |
| Aspartic acid | % | 0.12 | — |
| Methionine sulfon | % | 0.02 | 2 |
| Threonine | % | 0.08 | — |
| Serine | % | 0.08 | — |
| Glutamic acid | % | 0.14 | — |
| Proline | % | 0.07 | — |
| Glycine | % | 0.06 | — |
| Alanine | % | 0.08 | — |
| Valine | % | 0.04 | — |
| Isoleucine | % | 0.05 | — |
| Leucine | % | 0.11 | — |
| Tyrosine | % | 0.04 | — |
| Phenylalanine | % | 0.06 | — |
| Lysine | % | 0.04 | — |
| Histidine | % | <0.01 | — |
| Arginine | % | 0.03 | — |
| Total | % | 1.03 | — |

Remarks:
— = No remarks
1. Tryptophan was not reported since it was destroyed completely during hydrolysis.
2. Cysteic acid & Methionine sulfon: Molecular weight represent Cysteine & Methionine, respectively.

7.2 Crude dietary fiber includes hemicellulose, low molecular polysaccharides such as dextrin, and cell walls components such as β-(1-3)-glucans.

7.3 Vitamins. In the biomass of Tremella mesenterica CBS 123296 were founf vitamins A (Retinol), C (Ascorbic acid), E (α-Tocopherol), and vitamins of group B.

TABLE 17
Vitamin A and group B vitamins content in Tremella mesenterica
CBS 123296

| | Content, μg/g |
|---|---|
| Vitamins | dry weight |
| Vitamin A (Retinol) | 1000 |
| Vitamin B₁ (Thiamine) | 1.28 |
| Vitamin B₃ (Niacin) | 400.0 |
| Vitamin B₆ (Pyridoxine) | 0.8 |
| Vitamin B₇ (Biotin) | 0.1 |

Among vitamins of group B, determined by the microbiological method, based on the estimation of growth characteristics of sensitive auxotroph microorganisms, T. mesenterica biomass is especially rich in niacin and is rich in vitamin A.

7.4 Glucan and Glucuronoxylomannan in Tremella mesenterica (Cbs 123296)

Composition was determined by GC analysis (glucose, mannose and xylose content) of the whole Tremella mesenterica submerged biomass and of the fractions obtained after ultracentrifugation. Glucan completely precipitates at 120 000 g, glucuronoxylomannan (GXM) mostly remains in solution, although partly precipitates as well. It was found that GXM constitutes about 70% and Glucan about 30% of dry weight of the polysaccharide biomass.

Pure glucan without GXM was obtained by separation of the Tremella mesenterica treated by high power ultrasound. Ultrasound treated GXM is better soluble in water and does not precipitate in ultracentrifuge, thus pure β-glucan can be isolated after two re-precipitations. Separation of 1.2 g of Tremella mesenterica gave 400 mg of precipitate (glucan with ˜10% GXM) and 800 mg of soluble product (GXM).

In GC analysis GXM content was taken as (Man+Xyl)*1.3 (1.3 was used to compensate for GlcA and acetates). The structure of glucuronoxylomannan in the newly isolated Tremella mesenterica (CBS 123296) was found to be somewhat different from glucuronoxylomannan isolated from other Tremella mesenterica strains (not shown). Glucan was estimated from glucose content. To avoid reduction of glucuronolactone into Glc, hydrolysates were treated with 24% NH₃ prior to NaBH₄ reduction. Since glucan is not well soluble in the 3M TFA used for hydrolysis, other set of samples was treated with concentrated HCl (40° C., 1 h) before hydrolysis. This procedure leads to increased Glc recovery, but xylose partially degrades.

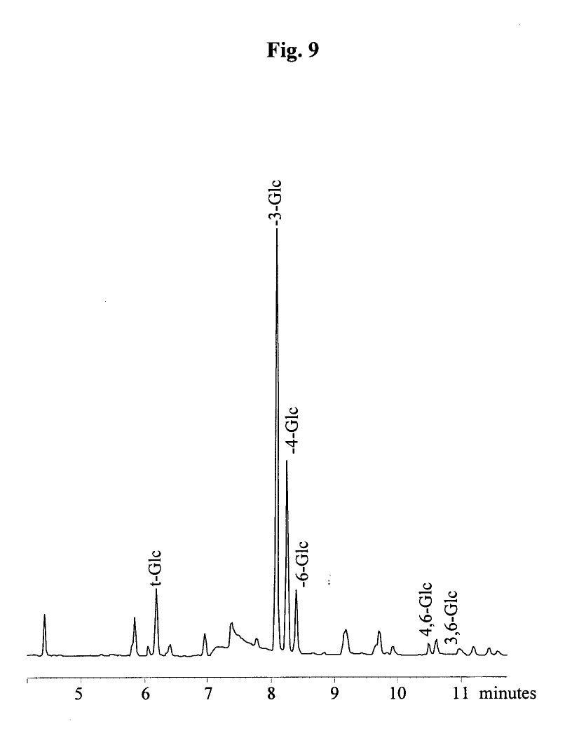

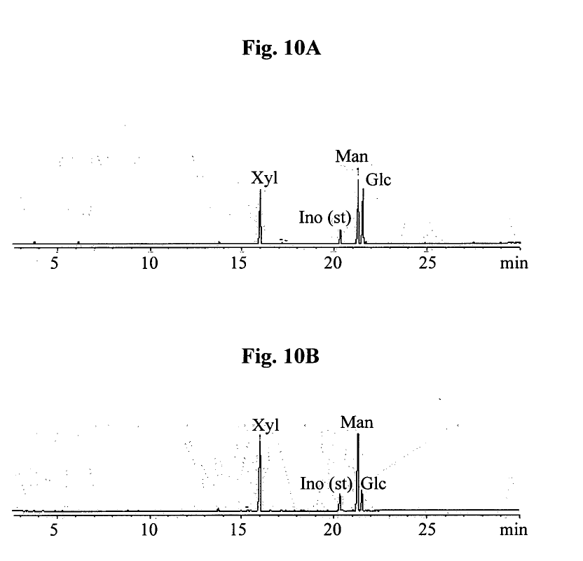

Methylation of the β-glucan showed that it has a linear structure with 3- and 4-substituted glucose as main components (FIG. 9 and FIG. 10). Thus the glucan is a 3,4-β-glucan. The glucan is not well soluble in DMSO, thus it was methylated twice; still some part was not dissolved, which may influence the results. The glucan is not soluble in water.

### Example 8

### Interferonogenous and Immuno-Modulating Properties of Tremella mesenterica CBS 123296 Preparations

Mongrel mice weighting 18-20 g were used in the study. The mice were fed per os by submerged single cell biomass of Tremella mesenterica CBS 123296.

The mice were killed after 6, 24 and 48 hours after treatment with the preparations, then blood plasma was collected in which the interferon (IFN) and tumors necrosis factor (TNF) levels were determined, and macrophages of peritoneal exudate aimed for examination of the phagocytosis system functional activity response to the preparations.

8.1 Plasma IFN Levels.

Interferogenic properties are presented in Table 18, and show that a single dose of biomass at 4 mg/animal given to mice per os induced synthesis of endogenous IFN in low titres (80 units/ml) after 24 hours. The IFN level in the blood serum of animals treated with the above dose of Tremella mesenterica biomass remained increased to 80 units/ml after 48 hours.

Tremella mesenterica biomass at a dose of 10 mg/animal appeared to be more active and caused generation of endogenous IFN in 80 units/ml titres 6 hours after administration. 24 hours after the preparation administration the IFN titres in the animal blood serum reached the maximum of 160-320 units/ml. Further (in 48 hours) the level of the serum IFN was reduced but still remained elevated (80 units/ml) in comparison with the control indices.

TABLE 18
Determination of interferonogenous activity of Tremella mesenterica
CBS 123296 biomass preparations in vivo

| | | IFN titre in units/ml | | |
|---|---|---|---|---|
| | | Time after the | | |
| | | preparation | | |
| | Dose | injection, hours | | |
| Preparation | mg/animal | 6 | 24 | 48 |
| Physiological | — | 20 | <20 | <20 |
| solution (control) | | | | |
| Poly I:C | 50 | 320 | 40 | <20 |
| T. mesenterica | 4 | 20 | 80 | 80 |
| biomass | 10 | 80 | 160-320 | 80 |

According to the tumor necrosis factor (TNF) level in the blood plasma determined in the study, Tremella mesenterica biomass did not result in synthesis of this cytokine in vivo (not shown). At the same time these preparations might assist in reducing the endogenous body intoxication. This is evidenced by the TNF detected in the blood plasma of control mice (at introduction of physiological solution). The use of the preparation results in reduced amount of TNF in blood plasma of control animals.

8.2 Response of the functional activity of macrophages of peritoneal exudate to Tremella mesenterica biomass. Functional activity of macrophages of peritoneal exudate was evaluated in Nitro Blue Tetrazolium (NBT)-test—the reaction of non-substrate reduction of nitro blue tetrazolium. The NBT test is commonly used for investigation of functional activity of macrophages, since the impaired NBT reduction ability of macrophages is concurrent with the pathology of their oxygen-dependent biocide properties. The activity of macrophages in NBT-test was examined without additional cells stimulation in vitro (spontaneous test) and with their stimulation in vitro with St. aureas cells (stimulated test). The stimulated NBT-test is regarded as a cytochemical criterion of the readiness to complete phagocytosis. The difference between the spontaneous and stimulated NBT-tests indices is regarded as cells functional reserve (FR), which reflects the effector potential of phagocytic system which fits well into the concepts about the general immunity reserves.

A single dose of Tremella mesenterica biomass at 4 and 10 mg/animal given per os was found to modify the oxygen-dependent biocide activity indices of macrophages of peritoneal exudate in spontaneous and stimulated NBT—tests (Table 19). It should be mentioned that the phagocyte activity response to Tremellastin was dependent on the dose and observation time. For example in 6 hours after a 4 mg/animal dose of Tremella mesenterica biomass was given, a slight drop of oxygen-dependent biocide activity and FR of macrophages occurred both in spontaneous and stimulated NBT-tests. Data presented in Table 19 show that after 24 and 48 hours the indices of the spontaneous NBT test increased reaching the control indices value in response to Tremella mesenterica biomass at 4 mg/animal. However, in comparison with the control a drastic increase of oxygen-dependent biocide activity of macrophage activity occurred in stimulated NBT-test during the given observation period (in 24 and 48 hours), which resulted in significant increase of their FR.

TABLE 19
Oxygen-dependent bactericidal activity of macrophages in vivo
response to Tremella mesenterica CBS 123296 biomass preparation

| | | Time | NVT-test | | |
|---|---|---|---|---|---|
| | | after | indices (%) | | |
| | Dose | injection, | Sponta- | Stimu- | Functional |
| Preparation | (mg/animal) | hours | neous | lated | reserve |
| Control | — | — | 55.2 | 64.6 | 9.4 |
| T. mesenterica | 4 | 6 | 44.5 | 47.5 | 3.0 |
| biomass | | 24 | 48.5 | 74.0 | 25.5 |
| | | 48 | 56.0 | 85.0 | 29.0 |
| T. mesenterica | 10 | 6 | 64.5 | 83.0 | 18.5 |
| biomass | | 24 | 56.6 | 75.8 | 19.8 |
| | | 48 | 56.0 | 94.0 | 38 |

In response to a single dose of 10 mg/animal of Tremella mesenterica biomass a slight increase of the indices of spontaneous NBT-test was observed after 6 hours, however after 24 and 48 hours the values reduced to the control index level. At the same time significant increase of macrophages activity was observed in stimulated NBT-test during the entire observation period—in 6, 24 and 48 hours. Increased stimulated NBT-test indices for mice macrophages treated with 10 mg/animal of Tremella mesenterica biomass resulted in drastic increase of FR cells. As follows from Table 19, FR increase occurred after 6 hours, and in 48 hours the maximum was attained.

As can be seen from our studies, Tremella mesenterica biomass had a minor influence on the indices of spontaneous NBT-test, however it caused significant increase of macrophages activity in stimulated NBT-test, in this way increasing the reserve potentialities of the phagocyte system.

As can be seen from the results of our investigation the oxygen-dependent bactericidal activity of macrophages in spontaneous NBT-test after 6 hours remained unchanged in comparison with the control. At the same time the stimulated NBT-test indices increased after 6 hours, which resulted in a significant increase of the cells FR. After 24 hours, the spontaneous NBT-test indices remained unchanged relative to the control, the indices of the stimulated test tended to increase and the FR of the cells remained unchanged too.

8.3 Summary. According to the investigation results analysis, a single dose of 4 and 10 mg/animal of Tremella mesenterica biomass preparation results in minor interferonogenous activity, inducing generation of “late” endogenous interferon. Tremella mesenterica biomass preparation appeared to be a more active interferonogen, moreover it was most efficient when concentrated to 10 mg/animal. In response to 10 mg/animal of Tremella mesenterica biomass preparation the peak value (160-320 units/ml) of interferon titres in blood serum was reached in 24 hours, which then decreased still remaining high during the entire period of observation. Tremella mesenterica biomass would be expected to prime, initiating generation of small amounts of interferon in the animals body, namely to intensify the interferon synthesis when injected together with other interferonogens.

Intensified functional activity of peritoneal exudate macrophages in the NBT-test in response to Tremella mesenterica biomass was observed. Tremella mesenterica biomass concentrated to 10 mg/animal appeared to be the most efficient activator of oxygen-dependent biocide activity of macrophages. The preparation caused an increase of the stimulated NBT-test indices (at unchanged indices of the spontaneous NT-test), which resulted in drastic increase of the functional reserve of the phagocyte system. It should be mentioned that the increase of the reserve potentialities of the phagocyte system is important to prevent the development of opportunistic bacterial or virus induced infections. It is also likely to prevent additional pathogenic-induced contamination of the body. Higher bactericidal activity of macrophages as well as higher functional reserve (with the difference between the spontaneous and stimulated NBT-test indices) accounted for the Tremella mesenterica biomass preparation correlated with interferon generation in the body. In this connection the activation of oxygen dependent macrophages biocide activity is associated with the induction of endogenous IFN by the Tremella mesenterica biomass (and possibly other cytokines, which needs to be further examined) since phagocytes are the main target of the IFN action in the body. Moreover, it is not improbable that phagocytes activated by the preparation are capable to take part in the generation of endogenous IFN by autocrine and paracrine activation technique.

### Example 9

### The Effect of Tremella mesenterica Cbs 123296 Extract and Glucuronoxylomannan on Plant Resistance to Phytoviruses

When chemical properties of polysaccharides isolated from the culture liquid and fruit bodies of Basidiomycetes were studied, we revealed neutral and acid polysaccharides. In particular, the acid polysaccharide of glucuronoxylomannan (GXM) produced by Tremella mesenterica consists of linear backbone of α-(1→3)-linked mannan, glycolized by β-(1→2)(1→4)-linked oligosaccharides of xylose and glucuronic acid, which gives the polyanion properties. Based on known data, it is possible to assume that neutral and acid glycans have different characteristics of antiphytoviral activity.

Indeed, the investigated polysaccharides differently inhibited the development of local lesions induced by TMV on Datura plants (Table 20).

Neutral polysaccharides proved to be the most active. The depression of the local lesions formation was up to 80 and 99.4% (in concentration of 100-1000 μg/mL). GXM was considerably less active, and, in this case, the total preparation occupied an intermediate position revealing evidence that the total preparation activity relative to infectivity of TMV is induced to a greater extent by neutral polysaccharides. The latter confirms the data from literature in which neutral polysaccharides relative to viral infectivity in supersensitive plants are more active than their sulfate derivatives, which mainly induce virus resistance of plants de novo. Based on the obtained results, it was important to investigate if GXM, which similar to sulfated mannan, can induce genetically dependent resistance of plants to viruses. It was found that GXM at a concentration of 1000-2500 μg/mL may induce resistance of the tobacco and datura plants to TMV (Table 21). In this case, AVR appeared to be higher in the tobacco plants than in datura plants. In other words, the activation of AVR by this polysaccharide depends on the genotype of the supersensitive host plant, and, consequently, on the activity of the proper gene of resistance.

TABLE 20
Influence of Tremella mesenterica CBS 123296 GXM and neutral
polysaccharides on infectivity of TMV in Datura stramonium plants

| | | Number of local | | |
|---|---|---|---|---|
| | Concentration | lesions per leaf | | |
| Polysaccharides | μg/mL | Experiment | Control | Inhibition, % |
| Total | 1000 | 0.5 | 11.9 | 96** |
| | 500 | 2.3 | 5.6 | 57* |
| Neutral | 1000 | 0.2 | 31.9 | 99.4* |
| | 100 | 0.8 | 4.1 | 80* |
| GXM | 1000 | 1.3 | 9.0 | 860 |
| | 100 | 4.5 | 5.2 | 16* |
| | 10 | 26.5 | 17.4 | −510 |

Note:
P0 > 5%;
*1% < P ≦ 5%;
**0.1% < P ≦ 1%;
*** P ≦ 0.1%

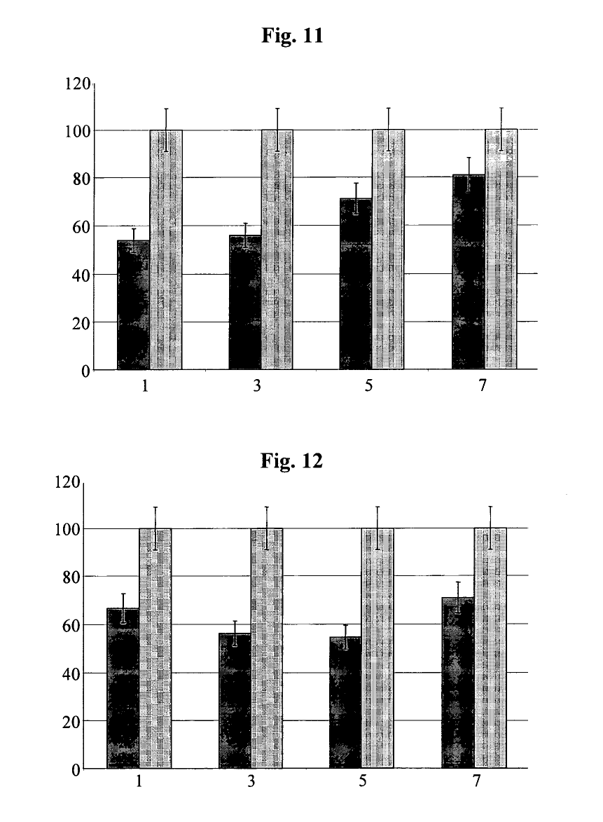

The investigation of the resistance development has shown that GXM at the concentration of 1000 and 2500 μg/mL induces the development of AVR relative to TMV in the tobacco plants already in the first day after inoculation with the inducer (FIGS. 11 and 12). For the concentration of GXM, which equaled 1000 μg/mL under the continued polysaccharide presence in plant tissues, the level of resistance gradually decreases: to 30%—in the 5th and to 20%—in the 7th days (FIG. 11). For the concentration of GXM, which equalled 2500 μg/mL, such a tendency is not observed (FIG. 12). On the contrary, this fact may provide evidence not only on the concentration dependence of polysaccharide induction of AVR at the gene level but also about the different ability of plants to adapt to lower and higher concentrations of GXM.

TABLE 21
Influence of glucuronoxylomannan on induction of resistance
against TMV-infection in Nicotiana tabacum and Datura
stramonium plants

| | | |
|---|---|---|
| | Number of local | |
| Concentration | lesions per leaf | Level of induced |

| | | | |
|---|---|---|---|
| μg/mL | Experiment | Control | resistance, % |

Nicotiana tabacum, variety Immune 580

| | | | |
|---|---|---|---|
| 100 | 98.1 | 100.0 | 20 |
| 500 | 102.7 | 104.7 | 20 |
| 1000 | 128.5 | 155.0 | 17* |
| 2000 | 63.4 | 133.1 | 52** |

Datura stramonium

| | | | |
|---|---|---|---|
| 100 | 71.2 | 79.8 | 110 |
| 500 | 90.0 | 99.3 | 90 |
| 1000 | 119.9 | 139.1 | 14* |
| 2000 | 119.6 | 181.1 | 33** |

Note:
see Table 20.

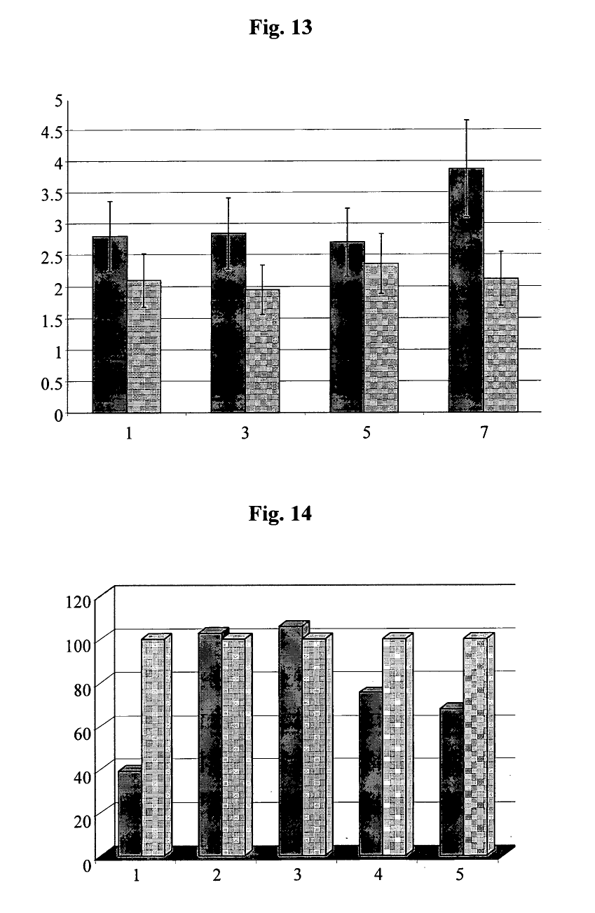

On measuring the sizes of TMV local lesions on the experimental and control halves of leaves, it was revealed that at the concentration of 2500 μg/mL GXM intensifies the growth of viral lesions (FIG. 13). At this concentration of GXM the reliable stimulation of the growth of necroses is observed only 7 days after inoculation with an inducer into the plant tissue, although such a tendency is observed in other periods of polysaccharide employment. On the halves of leaves treated with GXM at the concentration of 1000 μg/mL, reliable differences were not observed in the size of local lesions between the experiment and control.

It is known that similar influence on the growth of local necroses is produced by yeast RNA. This phenomenon may be explained by the activation in plants of the supersensitive mechanism of resistance under the influence of an inducer.

It was established in experiments with actinomycin D (AMD) that this antibiotic inhibits the development of GXM-induced virus resistance of plants (FIG. 14). AMD, irrespective of the method of its application (simultaneously or 2 days after introduction of GXM), inhibited the development of AVR induced with polyanion, either partly (20 μg/mL) or completely (10 μg/mL). Incomplete inhibition of AVR by actinomycin D at the concentration of 20 μg/mL may be caused by the antibiotic toxicity relative to plant tissues. The latter is confirmed by a decrease in the number of necroses in the control, where AMD was injected into leaves in the absence of GXM.

On the whole, the results obtained lead to the conclusion that AVR inoculated into supersensitive plants by GXM depends on the synthesis of new RNA on the matrices of cellular DNA. In other words, it is possible to attribute DNA to those inducers that activate plant resistance to viruses de novo.

Thus, it has been established that GXM induces resistance of supersensitive tobacco plants to the action of viral infection de novo, because this resistance is sensitive to the action of actinomycin D. On the one hand, its activity is similar to the action of previously studied sulphated polysaccharides and yeast RNA; on the other hand, the activity is similar to neutral polysaccharides that activate the supersensitive mechanism on the basis of the protein-carbohydrate interaction.

Taking into account that the mechanism of natural and induced resistance in plants, on the whole, has not been studied comprehensively, the search of substances capable of increasing natural virus resistance is promising. In our opinion the results of such studies have importance, as polysaccharides and also glycoproteins can be use in the future as a model of endogenous triggers that is involved in activation of AVR in supersensitive plants. Such substances will also be interesting for practical application with the purpose to decrease lesions of viral infections in agricultural and decorative plant growing.

1-21. (canceled)

22. New and distinct varieties of higher Basidiomycetes mushroom selected from Coprinus comatus HAI-1237 deposited under The Budapest Treaty with the Centralbureau voor Schimmelcultures (CBS) under Accession No. CBS 123401 (hereinafter Coprinus comatus CBS 123401), and Tremella mesenterica HAI-17 deposited under The Budapest Treaty with the Centralbureau voor Schimmelcultures (CBS) under Accession No. CBS 123296 (hereinafter Tremella mesenterica CBS 123296).

23. A biomass of a mushroom of claim 22 rich in nutraceutical agents and biologically active compounds including carbohydrates, proteins rich in essential amino acids, vitamins, lipids rich in essential fatty acids, antioxidant agents and minerals, wherein said biomass is obtained from the fruiting body or the mycelium of Coprinus comatus CBS 123401 or the mycelium of Tremella mesenterica CBS 123296.

24. The mycelial biomass of claim 23, wherein said carbohydrates of Coprinus comatus CBS 123401 include trehalose, β-glucans, and galactans, and said carbohydrates of Tremella mesenterica CBS 123296 include β-glucan.

25. An extract of a mushroom of claim 22 having nutraceutical and biological activity, obtained from the fruiting body or the mycelium of Coprinus comatus CBS 123401 or the mycelium of Tremella mesenterica CBS.

26. The extract of claim 25, wherein said extract is obtained from Coprinus comatus CBS 123401 culture, and is enriched in a low molecular weight water-soluble β-glucan and/or galactans, or said extract is obtained from Tremella mesenterica CBS 123296 culture, and is enriched in a linear 3,4 β-glucan and/or glucuronoxylomannan.

27. A composition comprising
a biomass of a mushroom comprising New and distinct varieties of higher Basidiomycetes mushroom selected from Coprinus comatus HAI-1237 deposited under The Budapest Treaty with the Centralbureau voor Schimmelcultures (CBS) under Accession No. CBS 123401 (hereinafter Coprinus comatus CBS 123401), and Tremella mesenterica HAI-17 deposited under The Budapest Treaty with the Centralbureau voor Schimmelcultures (CBS) under Accession No. CBS 123296 (hereinafter Tremella mesenterica CBS 123296);
an extract of a mushroom comprising new and distinct varieties of higher Basidiomycetes mushroom selected from Coprinus comatus HAI-1237 deposited under The Budapest Treaty with the Centralbureau voor Schimmelcultures (CBS) under Accession No. CBS 123401 (hereinafter Coprinus comatus CBS 123401), and Tremella mesenterica HAI-17 deposited under The Budapest Treaty with the Centralbureau voor Schimmelcultures (CBS) under Accession No. CBS 123296 (hereinafter Tremella mesenterica CBS 123296); or
a mixture of said biomass of (a) or extract of (b).

28. A pure submerged mycelial culture of Coprinus comatus CBS 123401.

29. A pure submerged mycelial culture of Tremella mesenterica CBS 123296, wherein said mycelial culture is in the form of single cell biomass.

30. A low molecular weight water-soluble β-glucan composed of a backbone structure of β-1-3-linked D-glucose residues bearing, at some of the 6-positions, 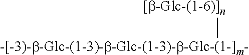
side chains of β-1-6-D-glucose residues, of the structure:
wherein m is an integer between 1 and about 10 and n is an integer equal to about 3.

31. The β-glucan of claim 30, obtained from Coprinus comatus.

32. A water insoluble linear 3,4 β-glucan obtained from Tremella mesenterica CBS 123296.

33. A glucuronoxylomannan obtained from Tremella mesenterica CBS 123296.

34. A composition comprising a carbohydrate selected from:
(a) the low molecular weight water-soluble β-glucan of claim 30;
(b) a water insoluble linear 3,4 β-glucan obtained from Tremella mesenterica CBS 123296;
(c) a glucuronoxylomannan obtained from Tremella mesenterica CBS 123296; or
(d) a combination of at least two carbohydrates of (a)-(c).

35. A food supplement, pharmaceutical, prebiotic, nutraceutical, beverage or cosmetic product comprising a composition of claim 27 or a composition comprising a carbohydrate selected from:
(a) the low molecular weight water-soluble β-glucan; composed of a backbone structure of β-1-3-linked D-glucose residues bearing, at some of the 6-positions, side chains 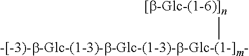
of β-1-6-D-glucose residues, of the structure:
wherein m is an integer between 1 and about 10 and n is an integer equal to about 3;
(b) a water insoluble linear 3,4 β-glucan obtained from Tremella mesenterica CBS 123296;
(c) a glucuronoxylomannan obtained from Tremella mesenterica CBS 123296; or
(d) a combination of at least two carbohydrates of (a)-(c).

36. A pet food, insecticidal, anti-parasitic or anti-plant virus product comprising a composition of claim 27 or a composition comprising a carbohydrate selected from:
(a) the low molecular weight water-soluble β-glucan; composed of a backbone structure of β-1-3-linked D-glucose residues bearing, at some of the 6-positions, side chains 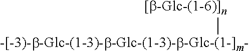
of β-1-6-D-glucose residues, of the structure:
wherein m is an integer between 1 and about 10 and n is an integer equal to about 3;
(b) a water insoluble linear 3,4 β-glucan obtained from Tremella mesenterica CBS 123296;
(c) a glucuronoxylomannan obtained from Tremella mesenterica CBS 123296; or
(d) a combination of at least two carbohydrates of (a)-(c).

37. A pharmaceutical composition comprising a pharmaceutically acceptable carrier and an active ingredient comprising
the composition of claim 27 or a composition comprising a carbohydrate selected from:
(a) the low molecular weight water-soluble β-glucan; composed of a backbone structure of β-1-3-linked D-glucose residues bearing, at some of the 6-positions, side chains 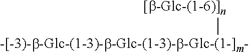
of β-1-6-D-glucose residues, of the structure:
wherein m is an integer between 1 and about 10 and n is an integer equal to about 3, preferably having a molecular weight of less than 10,000 Da, preferably about 1000 to about 10,000 Da;
(b) a water insoluble linear 3,4 β-glucan obtained from Tremella mesenterica CBS 123296;
(c) a glucuronoxylomannan obtained from Tremella mesenterica CBS 123296; or
(d) a combination of at least two carbohydrates of (a)-(c).

38. A method for treatment of diabetes; reduction of blood glucose levels; induction of an immunomodulatory response; reduction of blood cholesterol levels; or reduction of build up of cholesterol, said method comprising administering to a subject in need a therapeutically effective amount of the pharmaceutical composition of claim 37.

39. An agricultural composition comprising a carrier and an active ingredient selected from a composition of claim 27 or a composition comprising a carbohydrate selected from:
(a) the low molecular weight water-soluble β-glucan; composed of a backbone structure of β-1-3-linked D-glucose residues bearing, at some of the 6-positions, side chains 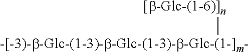
of β-1-6-D-glucose residues, of the structure:
wherein m is an integer between 1 and about 10 and n is an integer equal to about 3, preferably having a molecular weight of less than 10,000 Da, preferably about 1000 to about 10,000 Da;
(b) a water insoluble linear 3,4 β-glucan obtained from Tremella mesenterica CBS 123296;
(c) a glucuronoxylomannan obtained from Tremella mesenterica CBS 123296; or
(d) a combination of at least two carbohydrates of (a)-(c).

40. The agricultural composition of claim 39 for inducing resistance in plants to a plant virus.

41. The biomass of claim 23, wherein the mycelium of said Tremella mesenterica 123296 is from a pure submerged mycelium culture of the mushroom.
